(19) United States
(12) Patent Application Publication
(10) Pub. No.: US 2020/0206025 A1
(43) Pub. Date: Jul. 2, 2020

(54) Active Agent Delivery Devices and Methods for Using the Same

(71) Applicant: iRenix Medical, Inc., Palo Alto, CA (US)

(72) Inventors: Thomas W. Chalberg, Jr., Menlo Park, CA (US); Espir Gabriel Kahatt, Carlsbad, CA (US); Michael Edward Williams, San Diego, CA (US)

(21) Appl. No.: 16/622,170

(22) PCT Filed: Jun. 12, 2018
(86) PCT No.: PCT/US2018/037157

§ 371 Date: Dec. 12, 2019

Related U.S. Application Data
(60) Provisional application No. 62/519,091, filed on Jun. 13, 2017.
(60) Provisional application No. 62/533,967, filed on Jul. 18, 2017.

(51) Int. Cl. A61F 9/00

(52) U.S. Cl. CPC A61F 9/0008

(57) ABSTRACT

Active agent delivery devices are provided. Aspects of the devices include an active agent delivery component having an active agent delivery system; and an actuator component, where the active agent delivery component is release-ably engaged to the actuator component. The actuator component includes an active agent delivery system actuator. Either or both components may contain one or more elements of a pain mitigation system and/or a smart device system, e.g., an identifier and identifier reader. Also provided are methods of using the devices.

### CROSS-REFERENCE TO RELATED APPLICATIONS

This application is related to U.S. Provisional Patent Application Ser. No. 62/519,091 filed Jun. 13, 2017 and U.S. Provisional Patent Application Ser. No. 62/533,967 filed Jul. 18, 2017; the disclosures of which applications are herein incorporated by reference.

### INTRODUCTION

The parenteral administration (i.e., administration in a manner other than through the digestive tract, such as by intravenous, intramuscular or intraocular injection) of beneficial agents and fluids is an established clinical practice. Parenteral administration of beneficial agents provides effective remedies for many patients when administered properly.

The ocular surface is a tissue surface to which therapeutic agents may be parenterally delivered. The ability to deliver medication directly into the eye via intravitreal injection therapy (IVT) has transformed the treatment landscape of a number of previously blinding diseases, including macular degeneration and diabetic retinopathy. The success of these therapies in preventing blindness has resulted in a dramatic increase in the number of intravitreal injections performed, with an estimated 4.1 million injections given in the United States alone in 2013. The number of indications for IVT continues to expand, increasing utilization of this therapy significantly every year. The primary limitations of IVT are patient discomfort, ocular surface bleeding, corneal toxicity, and the time constraints of treating the vast number of patients requiring this therapy. These drawbacks relate to the difficulty of delivering ocular anesthesia to the highly vascularized ocular surface.

To give an ocular injection, the physician first provides ocular surface anesthesia by one or more of a number of methods, including the following: topical application of anesthetic drops; a subconjunctival injection of lidocaine; placement of cotton tipped applicators (commonly called a “pledget”) soaked in lidocaine over the planned injection site, application of topical anesthetic gel, or some combination of these. Following ocular anesthesia, the physician or an assistant sterilizes the periocular region by coating it in betadine or a similar antiseptic. Optionally, an eyelid speculum is placed, and the physician marks the location of the injection using calipers that guide placement of the needle. The ocular surface is again sterilized, and the physician gives the injection. Current methods of local anesthesia have unique drawbacks and patients often experience discomfort during and after intraocular injections.

### SUMMARY

Active agent delivery devices are provided. Aspects of the devices include an active agent delivery component having an active agent delivery system; and an actuator component, where the active agent delivery component is release-ably engaged to the actuator component. The actuator component includes an active agent delivery system actuator. Either or both components may contain one or more elements of a pain mitigation system and/or a smart device system, e.g., an identifier and identifier reader. Also provided are methods of using the devices.

### BRIEF DESCRIPTION OF THE FIGURES

FIGS. 1A and 1B provide assembled and disassembled views, respectively, of a device according to an embodiment of the invention that is configured to deliver a therapeutic agent to an ocular tissue delivery site.

FIG. 2 provides a view of a therapeutic agent delivery component according to an embodiment of the invention.

FIG. 3 provides side and back views of a therapeutic agent delivery system of a therapeutic agent delivery component according to an embodiment of the invention.

FIG. 4 provides an illustration of a reservoir of a therapeutic agent delivery component being filled with a therapeutic agent composition.

FIG. 5 provides a view of an actuator component according to an embodiment of the invention.

FIG. 6A provides a perspective view of a device according to another embodiment of the invention. FIG. 6B provides a side view of the device shown in FIG. 6A.

FIG. 7 provides a side view of a therapeutic agent delivery component release-ably engaged to a distal end of an actuator component according to an embodiment of the invention.

FIGS. 8A and 8B provide views of different sub-components of a therapeutic agent delivery component according to an embodiment of the invention.

FIGS. 9A and 9B provide views of a therapeutic agent delivery system according to an embodiment of the invention.

FIGS. 10A and 10B provide views of an actuator component according to an embodiment of the invention.

FIGS. 11A and 11B provide close up views of the distal end of an actuator component according to an embodiment of the invention.

FIG. 12 provides a view of a needle of a therapeutic delivery system engaged in the needle guide of a needle of an actuator component.

FIG. 13 provides a view of a system according to an embodiment of the invention

FIG. 14 provides a view of a docking station system that may be employed with the components of the delivery device shown in FIGS. 1A and 1B.

FIG. 15 provides a view of a docking station according to another embodiment of the invention.

FIGS. 16A to 16I provide depictions of various steps of a therapeutic agent delivery workflow according an embodiment of the invention.

### DEFINITIONS

As used herein, the term “tissue” refers to one or more aggregates of cells in a subject (e.g., a living organism, such as a mammal, such as a human) that have a similar function and structure or to a plurality of different types of such aggregates. Tissue may include, for example, organ tissue, muscle tissue (e.g., cardiac muscle; smooth muscle; and/or skeletal muscle), connective tissue, ocular tissue (e.g. conjunctival, episcleral, scleral, suprachoroidal, choroidal, retinal, subretinal, intravitreal, intracameral), nervous tissue and/or epithelial tissue.

An active agent is any component that provides pharmacological activity or other direct effect in the diagnosis, cure, mitigation, treatment, or prevention of disease, or affects the structure or any function of the body of man or animals. A type of active agent is a therapeutic agent, which are agents that may be used in treating, remediating, or curing a disorder or disease. Examples of active, e.g., therapeutic, agents include, but are not limited to steroids such as corticosteroids including dexamethasone (e.g., Ozurdex™), fluocinolone (e.g., Retisert™ or Iluvien™, loteprednol, difluprednate, fluorometholone, prednisolone, medrysone, triamcinolone, betamethasone and rimexolone; nonsteroidal anti-inflammatory agents such as salicylic-, indole acetic-, aryl acetic-, aryl propionic- and enolic acid derivatives including bromfenac, diclofenac, flurbiprofen, ketorolac tromethamine and nepafenac; antibiotics including azithromycin, bacitracin, besifloxacin, ciprofloxacin, erythromycin, gatifloxacin, gentamicin, levofloxacin, moxifloxacin, ofloxacin, sulfacetamide and tobramycin; VEGF inhibitors such as tyrosine kinase inhibitors, antibodies to VEGF, antibody fragments to VEGF, VEGF binding fusion proteins (e.g., pegaptinib, ranibizumab, bevacizumab, aflibercept, brolucizumab, Abicipar); PDGF inhibitors, antibodies to PDGF, antibody fragments to PDGF, PDGF binding fusion proteins (e.g., (Fovista™); anti-Factor D (lampalizumab), anti-TNF alpha agents such as antibodies to TNF-alpha, antibody fragments to TNF-alpha and TNF binding fusion proteins including infliximab, etanercept, adalimumab, certolizumab and golimumab; mTOR inhibitors such as sirolimus (Opsiria™) sirolimus analogues, Everolimus, Temsirolimus and mTOR kinase inhibitors; gases such as air, SF6, C2F6, C3F8, and others used in, for example, pneumatic retinopexy and retinal detachment repair; cells such as mesenchymal cells (e.g. mesenchymal stem cells), or cells transfected to produce a therapeutic compound; neuroprotective agents such as antioxidants, calcineurin inhibitors, NOS inhibitors, sigma-1 modulators, AMPA antagonists, calcium channel blockers and histone-deacetylases inhibitors; antihypertensive agents such as prostaglandin analogs, beta blockers, alpha agonists, and carbonic anhydrase inhibitors; aminosterols such as squalamine; antihistamines such as H 1-receptor antagonists and histamine H2-receptor antagonists; therapeutic cells; tyrosine kinase inhibitors and nucleic acid based therapeutics such as gene vectors, complement inhibitors; chemotherapeutic agents; insulin; plasmids and siRNA.

An active agent composition is a composition that includes one or more active agents. A therapeutic agent composition is such a composition where the active agent thereof is a therapeutic agent. Active agent delivery devices are devices that may be employed to deliver an active agent or composition thereof to a subject, where a therapeutic agent delivery device is a such a device where the active agent or composition thereof is a therapeutic agent. Active agent delivery components are delivery components of active agent delivery devices, where a therapeutic agent delivery component is a such a component where the active agent or composition thereof is a therapeutic agent.

The term “subject” is used interchangeably in this disclosure with the term “patient”. In certain embodiments, a subject is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, subjects are humans. The term “humans” may include human subjects of both genders and at any stage of development (e.g., fetal, neonates, infant, juvenile, adolescent, adult), where in certain embodiments the human subject is a juvenile, adolescent or adult. While the devices and methods described herein may be applied to perform a procedure on a human subject, it is to be understood that the subject devices and methods may also be carried out to perform a procedure on other subjects (that is, in “non-human subjects”).

In some instances, the devices or portions thereof may be viewed as having a proximal and distal end. The term “proximal” refers to a direction oriented toward the operator during use or a position (e.g., a spatial position) closer to the operator (e.g., further from a subject or tissue thereof) during use (e.g., at a time when a tissue piercing device enters tissue). Similarly, the term “distal” refers to a direction oriented away from the operator during use or a position (e.g., a spatial position) further from the operator (e.g., closer to a subject or tissue thereof) during use (e.g., at a time when a tissue piercing device enters tissue). Accordingly, the phrase “proximal end” refers to that end of the device that is closest to the operator during use, while the phrase “distal end” refers to that end of the device that is most distant to the operator during use.

Modules are made up of one or more functional blocks which act in concert to perform a particular function, which is the purpose of the module. A given module may be implemented as hardware, software or a combination thereof. In some instances, modules may include a circuitry element, such as an integrated circuit. When present, integrated circuits may include a number of distinct functional blocks, where the functional blocks are all present in a single integrated circuit on an intraluminal-sized support. By single integrated circuit is meant a single circuit structure that includes all of the different functional blocks. As such, the integrated circuit is a monolithic integrated circuit (also known as IC, microcircuit, microchip, silicon chip, computer chip or chip) that is a miniaturized electronic circuit (which may include semiconductor devices, as well as passive components) that has been manufactured in the surface of a thin substrate of semiconductor material.

Furthermore, the definitions and descriptions provided in one or more (e.g., one, two, three, or four, etc.) sections of this disclosure (e.g., the “Descriptions”, “Devices”, “Methods” and/or “Kits” sections below) are equally applicable to the devices, methods and aspects described in the other sections.

### DETAILED DESCRIPTION

Active agent delivery devices are provided. Aspects of the devices include an active agent delivery component having an active agent delivery system; and an actuator component, where the active agent delivery component is release-ably engaged to the actuator component. The actuator component includes an active agent delivery system actuator. Either or both components may contain one or more elements of a pain mitigation system and/or a smart device system, e.g., an identifier and identifier reader. Also provided are methods of using the devices.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which may be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Devices

As summarized above, active agent delivery devices are provided. As the devices are active agent delivery devices, they are configured to deliver an amount (e.g., dosage) of an active, e.g., therapeutic, agent to a target tissue delivery site of a subject. As will be reviewed in greater detail below, the active agent may be in any convenient state, such as liquid, solid or semi-solid (e.g., gel), or gas. In some embodiments, the devices have integrated pain mitigation, such that they are configured to alleviate pain associated with delivery of the active, e.g., therapeutic agent, to the target tissue delivery site by the device. While the magnitude of pain mitigation may vary, in some instances the magnitude of pain mitigation is 5% or more, such as 10% or more, and including 20% or more, as compared to a suitable control (such as identical delivery without pain mitigation). In some embodiments, the devices are “smart”, such that they include one or more electronic components that are configured to provide for wireless communication at least between an active agent delivery component and an actuator component of the device, if not with one or more additional networked devices.

Aspects of the devices include an active agent delivery component having an active agent delivery system and an actuator component, where the active agent delivery component is release-ably engaged to the distal end of the actuator component and the actuator component includes an active agent delivery system actuator. As the active agent delivery component is release-ably engaged to the actuator component (e.g., to the distal end of the actuator component), it is detachable from the actuator component. Accordingly, the active agent delivery component is configured to be readily separable from the actuator component without in any way damaging the functionality of the actuator component, such that actuator component may be attached to another active agent delivery component to produce a complete active, e.g., therapeutic, agent delivery device of the invention. As such, the devices of the present invention are configured so that the actuator component can be sequentially operably attached to multiple different active agent delivery components. Of interest are configurations in which the active agent delivery component can be manually operably attached to the actuator component unit without the use of any tools. A variety of different configurations may be employed, e.g., where the proximal end of the active agent delivery component engages the distal end of the actuator component to provide an operable connection between the two, such as by a snap-fit configuration, an insertion and twist configuration, luer-lock configuration, etc. In certain configurations, the actuator component has a structure configured to receive the proximal end of the elongated member. In some instances, the device further includes a locking element for release-ably engaging the active agent delivery component to the distal end of the actuator component of the device. Any convenient locking mechanism may be employed, such as but not limited to: luer lock, screw on, and the like.

As reviewed above, the active agent delivery devices may include an integrated pain mitigation system. Accordingly, aspects of the devices may further include a pain mitigation system, the components of which may be present in either or both of the active agent delivery component and the actuator component. A pain mitigation system is a system that provides for pain alleviation during delivery of a an active, e.g., therapeutic, agent to a target delivery site, as discussed above. The pain mitigation system may vary as desired, where pain mitigation systems finding use in devices of the invention include both anesthesia producing systems (i.e., systems that result in at least some degree of, if not complete loss of, sensation in the target tissue delivery site, e.g., via blockage of all feeling in the target tissue delivery site) and analgesia producing systems (i.e., systems that result in relief of pain without total loss of feeling in the target tissue delivery site).

In some instances, an anesthesia producing system is a cooling system, i.e., a system that decreases the temperature of the target tissue delivery site by an amount sufficient to produce the desired anesthesia in the target tissue delivery site. The cooling system may vary, and in some instances is a system that provides for contact of a cold element (e.g., a cold tip or cold tissue engager) with the target tissue delivery site. The cold element (which may be a component of a tissue engager, e.g., as described in greater detail below) of the cooling system may vary, and in some instances is an element that is configured to maintain a temperature of between −80° C. to +5° C., such as −20° C. to 0° C. and including −10° C. to −5° C. when contacted with the target tissue delivery site. During a given delivery method, a tissue engager may maintain a constant temperature or cycle through one or more distinct temperature ranges, as desired. For example, a tissue engager may be configured to have a temperature that falls within a first range (e.g., as described above) to provide for desired cryoanesthesia during active, e.g., therapeutic, agent delivery, and then cycle to a second, warmer temperature prior to remove of the device, such as a temperature ranging from 0 to −5, such as 0 to −2.5, including 0 to −1° C. Where the target tissue delivery site is an ocular tissue delivery site, e.g., as described elsewhere, delivery of cooling to cause rapid vasoconstriction enables a reduction in the occurrence of ocular surface bleeding and prevents repeated vascular trauma with long term circulatory compromise.

Specific cooling systems of interest that may find use in anesthesia producing pain mitigation systems may vary, where cooling systems of interest include, but are not limited to: thermoelectric cooling systems, liquid evaporation cooling systems, solid sublimation cooling systems, Joule-Thompson cooling systems, thermodynamic cycle cooling systems, endothermic reaction cooling systems and a low-temperature substance cooling systems, and the like.

In some instances, the pain mitigation anesthesia producing system is a thermoelectric cooling system, e.g., one that includes one or a combination of thermoelectric (Peltier) devices. While thermoelectric cooling systems employed in embodiments of devices of the invention may vary, in some instances the thermoelectric cooling systems include a cold tip that is configured to contact a target tissue delivery site (and therefore may also be referred to as a tissue engager), a power source, a controller, a cooling power concentrator, one or more Peltier unit modules, and a heat sink. It should be understood that, in some embodiments, a given thermoelectric cooling system may include a heating element (not shown) that operates in conjunction with the cooling elements to precisely maintain a desired temperature and/or heat flux. Further details regarding embodiments of thermoelectric cooling systems that may be employed in devices of the invention are provided in U.S. Published Patent Application Publication No. 20160279350; the disclosure of which is herein incorporated by reference.

As reviewed above, other non-thermoelectric cooling pain mitigation systems may be employed, such as but not limited to: liquid evaporation cooing system, solid sublimation cooling system, Joule-Thompson cooling system, thermodynamic cycle cooling system, an endothermic reaction cooling system and a low-temperature substance cooling system.

Instead of cooling systems, other types of anesthesia producing systems may be employed as pain mitigation systems. Such anesthesia systems include, but are not limited to: system that deliver an anesthetic agent, such as but not limited to: sodium-channel blockers, e.g., as amino amides or animo esters, i(such as proparacaine, tetracaine, or lidocaine drops, gels, or creams), naturally-derived agents, such as saxitoxin, neosaxitoxin, tetrodotoxin, menthol, eugenol, and cocaine; and the like.

Also of interest as pain mitigation systems are analgesia producing systems, e.g., as summarized above. Examples of analgesia producing systems finding use in embodiments of devices of the invention include application of agents considered above as local anesthetics. They may also include, but are not limited to additional techniques such as electrical stimulation (Campbell and Taub, Arch Neurol. 1973; 28(5):347-350.) and the like.

Active agent delivery devices as described herein may be handheld. In such embodiments, as the devices are handheld, they are configured to be held easily in the hand of an adult human. Accordingly, the devices may have a configuration that is amenable to gripping by the human adult hand. The weight of the devices may vary, and in some instances may range from 0.05 to 3 pounds, such as 0.1 pounds to 1 pound. Handheld devices of the invention may have any convenient configuration, where examples of suitable handle configurations are further provided below.

The active agent delivery devices of the invention may include one or more sensors. When present, the one or more sensors may be part of the active, e.g., therapeutic, agent delivery component and/or the actuator component. The sensors may vary, where sensors may rely on mechanical, electrical or chemical sensing mechanisms. When present, sensors may be used to obtain a variety of different types of information, such as historical information, use information, etc., (e.g., as described in greater detail below), which information may be incorporated into an identifier, transmitted to a networked device, etc., as described in greater detail below.

The active agent delivery devices of the invention may be configured for delivery of an active agent to a variety of target tissue delivery sites. Examples of target tissue delivery sites include both external and internal delivery sites, wherein internal delivery sites include those sites located in body cavities. External sites may include keratinized sites, as well as sites characterized by cutaneous membranes, mucous membranes, and tissue of the mucocutaneous zone. In some instances, the target tissue delivery site is an ocular tissue delivery site, where ocular tissue delivery sites of interest include a region that begins at the corneal limbus and extends anywhere from 1 mm to 10 mm posterior to the limbus, 2 mm to over 8 mm posterior to the limbus, such as 3 mm to 6 mm from the corneal limbus, e.g., 3 to 4 mm from the corneal limbus, e.g., to allow intraocular injection via pars plana or pars plicata. Ocular tissue delivery sites may include conjunctiva, episclera, and sclera of the eye. Ocular tissue delivery sites of interest include those that provide for intravitreal injection therapy (IVT), retrobulbar injection therapy, subtenon injection therapy, subretinal injection therapy, suprachorodial injection, subconjunctival injection therapy, intracameral injection therapy, and the like.

As summarized above, delivery devices of the invention include an active agent delivery component that is release-ably engaged to an actuator component. Each of these components of the device is now described separately in greater detail.

### Active Agent Delivery Component

The active agent delivery component includes an active agent delivery system and is configured to be release-ably engaged to an actuator component of the device, e.g., as described above. Active agent delivery components have a body having a distal, tissue contacting end and a proximal actuator component engaging end. The dimensions of the distal tip of the active agent delivery component may vary, where in some instances the distal tip has a longest dimension ranging from 1 to 10 mm, such as 3 to 7 mm, e.g., 5 mm. The shape of the body may also vary as desired, where examples of different shapes are provided below in conjunction with the description of specific embodiments of the device. In some instances, the dimension of the distal end of the tip will be such that the injector, e.g., needle, entry occurs at a predetermined distance from the corneal limbus (where distances may be as provided above) when the tip is placed on the ocular surface, where such configurations may negate any need for manual measurement for safe injection distance prior to procedure (intravitreal, intracameral etc.). While the weight of the active agent delivery component may also vary, active agent delivery component has a weight ranging from 0.25 pounds to 1 pound, such as 0.1 pounds to 0.7 pounds, where in some instances the weight is 0.5 pounds or less, such as 0.3 pounds or less.

Located in the body of the active agent delivery component is an active agent delivery system. The active agent delivery system is a system that, upon actuation by an actuator of the actuator component, delivers an amount, e.g., a dosage, of an active agent to a target tissue delivery site. The active agent composition that is delivered to the target tissue delivery site may be a composition that is in a variety of different physical states, including liquid, solid, semi-solid (e.g., gel) and gaseous. As such, the active agent delivery system may vary depending on the physical state of the active, e.g., therapeutic, agent composition.

Active agent delivery systems are systems that include one or more components of an active agent composition delivery mechanism, which mechanism may vary depending on the nature of the active agent composition and how the disparate components of the mechanism are distributed among the active agent delivery component and the actuator component.

Where the active agent composition is a flowable composition, e.g., a composition having a liquid or semi-solid (e.g., gel) state (such as where the viscosity ranges from 1 to 10,000,000 cps), the active agent delivery system may include one or more components of a delivery mechanism that is configured for delivery of the flowable composition. Examples of components that may be present in the active agent delivery system include an injector, such as a needle or cannula, or at least a passage way therefor, a reservoir, e.g., in those embodiments where the reservoir is not located in the actuator, etc. In some instances, the active agent delivery system of the active agent delivery component includes an active agent reservoir operably engaged with a tissue injector, where the tissue injector is a structure configured to convey the flowable active agent composition from the reservoir to the target tissue delivery site.

The active agent reservoir may be a compartment of any convenient configuration. While the volume of the reservoir may vary, in some instances the volume ranges from 0.01 mL to 10 mL, such as 0.1 mL to 5 mL. For embodiments configured for ocular delivery, volumes may vary, and in some instances range from 0.01 mL to 1 mL, such as from 0.05 mL to 0.15 mL. In some instances, the volume of the reservoir is sufficient to hold an amount of an active agent composition that is greater than the amount which is delivered to a target tissue delivery site during use of the device. While the magnitude of the excess may vary, in some instances the magnitude ranges from 110% to 500% of the delivered volume, such as 120% to 150%. Where the active agent composition is a non-gaseous composition, the amount of any gas, e.g., air, in the reservoir (and other components of the active agent delivery system, may be minimal, where in some instances the amount is 10 μl or less, such as 5 μl or less, 3 μl or less, 2 μl or less, or 1 μl or less.

The active agent reservoir may include an active agent composition, i.e., a composition that includes one or more active agents and one or more delivery vehicle components, e.g., water, buffers, preservatives, etc. As reviewed above, an active agent is any component that provides pharmacological activity or other direct effect in the diagnosis, cure, mitigation, treatment, or prevention of disease, or affects the structure or any function of the body of man or animals. A type of active agent is a therapeutic agent, which are agents that may be used in treating, remediating, or curing a disorder or disease. Examples of active, e.g., therapeutic, agents include, but are not limited to steroids such as corticosteroids including dexamethasone (e.g., Ozurdex™), fluocinolone (e.g., Retisert™ or Iluvien™), loteprednol, difluprednate, fluorometholone, prednisolone, medrysone, triamcinolone, betamethasone and rimexolone; nonsteroidal anti-inflammatory agents such as salicylic-, indole acetic-, aryl acetic-, aryl propionic- and enolic acid derivatives including bromfenac, diclofenac, flurbiprofen, ketorolac tromethamine and nepafenac; antibiotics including azithromycin, bacitracin, besifloxacin, ciprofloxacin, erythromycin, gatifloxacin, gentamicin, levofloxacin, moxifloxacin, ofloxacin, sulfacetamide and tobramycin; VEGF inhibitors such as tyrosine kinase inhibitors, antibodies to VEGF, antibody fragments to VEGF, VEGF binding fusion proteins (e.g., pegaptinib, ranibizumab, bevacizumab, aflibercept, brolucizumab, Abicipar); PDGF inhibitors, antibodies to PDGF, antibody fragments to PDGF, PDGF binding fusion proteins (e.g., (Fovista™); anti-Factor D (lampalizumab), anti-TNF alpha agents such as antibodies to TNF-alpha, antibody fragments to TNF-alpha and TNF binding fusion proteins including infliximab, etanercept, adalimumab, certolizumab and golimumab; mTOR inhibitors such as sirolimus (Opsiria™) sirolimus analogues, Everolimus, Temsirolimus and mTOR kinase inhibitors; gases such as air, SF6, C2F6, C3F8, and others used in, for example, pneumatic retinopexy and retinal detachment repair; cells such as mesenchymal cells (e.g. mesenchymal stem cells), or cells transfected to produce a therapeutic compound; neuroprotective agents such as antioxidants, calcineurin inhibitors, NOS inhibitors, sigma-1 modulators, AMPA antagonists, calcium channel blockers and histone-deacetylases inhibitors; antihypertensive agents such as prostaglandin analogs, beta blockers, alpha agonists, and carbonic anhydrase inhibitors; aminosterols such as squalamine; antihistamines such as H 1-receptor antagonists and histamine H2-receptor antagonists; therapeutic cells; tyrosine kinase inhibitors and nucleic acid based therapeutics such as gene vectors, complement inhibitors; chemotherapeutic agents; insulin; plasmids and siRNA.

Where desired, the active agent reservoir may include a port that provides for access to the inside of the reservoir from the outside of the active agent delivery component, e.g., to provide for introduction of an active agent composition into the reservoir prior to use. The port, when present, may have any suitable configuration. In some instances, the active agent delivery component may include a window configured to allow viewing of the reservoir from a location external to the active agent delivery component. For example, a window may be provided to allow for human viewing of the reservoir contents from a position external to the active agent delivery component, e.g., to confirm that the active agent delivery component is ready for use, e.g., includes a proper amount of an active agent composition in the reservoir. The window may have any suitable dimensions, and in some instances has an area ranging from 10 mm² to 1000 mm², such as 50 mm² to 500 mm². The window may be fabricated from any convenient transparent material, including glasses, plastics, etc.

In addition to a reservoir, the active agent delivery system may also include tissue injector. As summarized above, the tissue injector is an element configured to convey the active agent composition from the reservoir to, and in some instances into, a target tissue delivery site. In some instances the injector is a needle or cannula. The injector may have any convenient dimensions, and in some instances is has a gauge ranging from 20 to 35, such as 27 to 35, including 28 to 35, such as 29, 29½, 30, 31, 32, 33 and 34 gauge. The injector may be made of any convenient material, e.g., stainless steel, etc. Alternatively, the tissue injector may be needleless, such as a jet injector.

In some instances, the injector is configured to prevent contact with a tissue structure near a target tissue delivery site, e.g., an ocular lens or ocular retina where the target tissue delivery site is an ocular tissue delivery site. For example, the injector may be configured to extend only a certain limiting distance beyond the target tissue contacting end of the body of the tissue delivery component. While this limiting distance may vary, in some instances the limiting distance ranges from 0.5 to 8 mm, such as 3 to 4 mm. Where the active agent delivery system includes an injector, such as a needle or cannula, the distal, tissue contacting end of the active agent delivery component may include an opening dimensioned to provide for exit of the injector from the body during active agent delivery, thereby providing access of the active agent delivery system directly to a target tissue and, e.g., minimizing the potential for unwanted particles or other elements to be introduced into the target tissue. While the dimensions of the opening, when present, may vary, in some instances the opening has a diameter sufficient to accommodate passage of a needle having a gauge, e.g., as described above.

In some instances, the active agent delivery system may include one or more filters. The filters may be configured to remove particles or other unwanted components present in the active, e.g., therapeutic, agent composition prior to delivery to the target tissue delivery site. Such filters may be configured to inhibit passage of particles above a certain pore size from >0.1 μm to >50 μm, such as >5 μm. The one or more filters may be positioned at any convenient location in the active agent delivery system, e.g., at the exit from the reservoir into the injector, at some point along the injector, at the distal end of the injector, etc.

As summarized above, an active agent delivery component may include a pain mitigation system or component thereof. Pain mitigation systems of the devices of the invention may include a tissue engager, such as a cool tip. In such devices, the tissue engager may be integrated with the active agent delivery component, or separate from the active agent delivery component. When the tissue engager is separate from the active agent delivery component, the active agent delivery component may, where desired, may include a passage configured to accommodate the tissue engager, such as a passage extending from the proximal, actuator component engaging end to the distal, tissue contacting end of the body of the active agent delivery component. In some instances, the distal opening of the passage that provides for contact of the tissue engager with the target tissue delivery site is at least partially covered by a membrane that is displaced upon occupation of the passage by the tissue-engager. When present, this membrane may have a thickness ranging from 0.01 to 1 mm, and be fabricated from any convenient material.

Where desired, the distal, tissue contacting end of the active agent delivery component may include a removable cover, e.g., that is present until the device is used to deliver active, e.g., therapeutic, agent to a target delivery site. The cover may be configured as a release liner or analogous structure, such that it may be easily removed just prior to use. The cover may be sterile or sanitized as desired, and fabricated from any convenient material, e.g., plastics, etc. It may also take the form of a sterile peel pack, sterile box, etc.

The active agent delivery component may, where desired, include an antimicrobial element. The antimicrobial element may be any convenient element having antimicrobial properties and be positioned at one or more locations of the active agent delivery component. For example, the antimicrobial element may be positioned at the distal, tissue contacting end of the body in order to provide for at least aseptic conditions during contact of the device to the target tissue delivery site, in order to sanitize the target tissue delivery site, etc. The antimicrobial element may include an antimicrobial agent, which may be present in a holder, such as a matrix material, reservoir, etc. As with the active, e.g., therapeutic, agent, the antimicrobial agent, when present, may be present in a composition that is in a variety of different physical steps, including liquid, solid, semi-solid, and gaseous. Antimicrobial agents of interest include, but are not limited to: povidone-iodide (Betadine), chlorhexidine (Nolvasan), ethanol or other alcohols, and the like.

The active agent delivery component may, where desired, include an analgesic/anesthetic agent. When present, the analgesic/anesthetic agent may be present in any convenient manner that provides for delivery of the analgesic/anesthetic agent to the target tissue delivery site during use of the device. For example, the analgesic/anesthetic agent may be positioned at the distal, tissue contacting end of the body in order to provide for at least aseptic conditions during contact of the device to the target tissue delivery site. The analgesic/anesthetic agent may be present in a holder, such as a matrix material, reservoir, etc. As with the active agent, the analgesic/anesthetic agent, when present, may be present in a composition that is in a variety of different physical steps, including liquid, solid, semi-solid, and gaseous. Analgesic/anesthetic agents of interest include, but are not limited to: lidocaine, benzocaine, prilocalne, lidocaine, dubicaine, mepivacaine, bupivacaine, and the like; naturally-derived products, such as saxitoxin, neosaxitoxin, tetrodotoxin, menthol, eugenol, and cocaine, and the like; etc.

As indicated above, in some instances, the active agent delivery component may include a component of a locking element for release-ably engaging the active agent delivery component to the actuator component of the device, such as the distal end of the actuator component of the device. As reviewed above, any convenient locking mechanism may be employed, such as but not limited to: luer-lock, screw on, and the like. The locking element component of the locking element that is present on the active agent delivery component may vary, as desired, and is selected based on the companion element that is present on the actuator.

In some instances, the active agent delivery component further includes one or more identifiers. In some instances, an identifier present on the active agent delivery component is an identifier that is configured to be read by an identifier reader of the actuator component of the device. While such reader compatible identifiers may vary, in some instances the identifier is a barcode, such as a linear barcode or a matrix barcode, such as a QR code. In some instances, the reader compatible identifier is a radio frequency identification (RFID) tag, such as a near field communication (NFC) tag, where the RFID tag may be passive or active. Information included in the identifier may include, but is not limited to, identity of the active, e.g., therapeutic, agent (brand name and/or generic name), date of manufacture, date of expiry, source of manufacture, dosage amount, drug concentration, intended route of administration, handling and storage information, delivery volume, indication for use, lot number, etc.

In addition to, or instead of, a reader compatible identifier, the active agent delivery device may include an identifier that is visual identifier, such that it is configured to be read by a health care practitioner. Visual identifiers are identifiers that may be readily understood by a human upon looking at the identifier, such that computer processing of the identifier is not required. Examples of such identifiers include, but are not limited to, text identifiers, color coding identifiers, commonly understood symbols, identifying trademarks, logos, and the like. Information conveyed by the visual identifier may vary as desired, where examples of information that may be conveyed by the visual identifier include, but are not limited to: information about the active, e.g., therapeutic, agent delivery component or active, e.g., therapeutic, agent present therein, such as identity of the active, e.g., therapeutic, agent (brand name and/or generic name), date of manufacture, date of expiry, source of manufacture, dosage amount, drug concentration, intended route of administration, handling and storage information, delivery volume, indication for use, lot number, etc.

Information included in the identifier may include, but is not limited to, active, e.g., therapeutic, agent delivery component historical information, which information may include one or more of: an active, e.g., therapeutic, agent identifier (e.g., identity of the therapeutic agent (brand name and/or generic name); active, e.g., therapeutic agent dose, concentration and/or volume information; manufacturing information, such as manufacturing date, lot number and/or source for the active, e.g., therapeutic, agent delivery component or an active, e.g., therapeutic, agent composition thereof; active, e.g., therapeutic, agent delivery component handling information; active, e.g., therapeutic, agent delivery component and/or active, e.g., therapeutic, agent composition thereof expiration date. Other types of information that may be present on the identifier include, but are not limited to: administration information, therapeutic indication information, intended patient information, and the like.

The entire active agent delivery component may be configured for single use, such that the entire active agent delivery component is disposable. Alternatively, one or more components of the active agent delivery component may be reusable. For example, the body of the active agent delivery component may be reusable, e.g., where the body may be sterilized, such that the component may be used multiple times, e.g., by reloading active, e.g., therapeutic, agent into the active agent delivery system.

The active agent delivery components of the invention may be fabricated using any convenient materials or combination thereof, including but not limited to: metallic materials such as tungsten, copper, stainless steel alloys, platinum or its alloys, titanium or its alloys, molybdenum or its alloys, and nickel or its alloys, etc.; polymeric materials, such as polytetrafluoroethylene, polyimide, PEEK, and the like; ceramics, such as alumina (e.g., STEATITE™ alumina, MAECOR™ alumina), etc. The drug reservoir can be made of plastic, such as polypropylene or polystyrene, or any material commonly used for syringes and the like. It can also be made of glass, including type 1 glass, as is commonly used for long-term storage of drugs and biologics. Alternatively, it can be made of non-leachable plastic materials that are used for long-term storage of drugs or biologics, such as cyclic olefin polymer (such as Crystal Zenith), cyclic olefin co-polymer and the like.

In some instances, the active agent delivery component information includes a linked sensor. A linked sensor may be configured to detect a variety of different types of data, such as environmental data, e.g., temperature, moisture, exposure to radiation, and the like; locational data; etc. The sensor may be operably connected to a storage medium, such that data of interest may be recorded over a desired period of time, such as from manufacture of the component up to use of the component, or some portion thereof. Such data can be used in a variety of difference applications, such as described below.

### Actuator Component

Also present in the devices of the invention is an actuator component. As described above, the actuator component is configured to release-ably engage with an active agent delivery component, such as described above, to produce an active agent delivery device of the invention. Aspects of actuator components according to embodiments of the invention include a body having a proximal end and a distal end, wherein the distal end is configured to be release-ably engaged to an active agent delivery component, e.g., as described above. Further aspects of the actuator component include an active agent delivery system actuator configured to actuate an active agent delivery system of the active agent delivery component. In some instances, the actuator further includes one or more components of a pain mitigation system configured to mitigate pain in target tissue delivery site, and in some instances the actuator may include all of the components of a pain mitigation system.

The active agent delivery system actuator is an element or subsystem that is configured to actuate the active, e.g., therapeutic, agent delivery system of the active agent delivery component so as to deliver an active agent to a target tissue delivery site. The nature of the active agent delivery system actuator may vary, e.g., depending on the nature of the active agent delivery system. For example, where the active agent delivery system includes a reservoir operably engaged to a tissue injector, the active agent delivery system actuator may be configured to provide for control of one or more of angular position, linear position, velocity and acceleration of the tissue injector. In some instances, the actuator, either alone or in conjunction with a guiding element of the active agent delivery component, is configured to provide for an angle of the injector relative the distal, tissue contacting end of the active agent delivery component, that ranges from 0 to 90, such as 75 to 90°. In some embodiments, the active agent delivery system is present in the device at a pre-determined angle, for example, 90 degrees to the biologic tissue when the cold tip is applied to the biologic tissue, so that when the device tip is placed on the eye abutting the limbus and causing very slight indentation of the ocular surface 360 degrees around the tip, the needle tip will reproducibly be inserted into the eye at a defined, safe angle posterior to the limbus of the eye to avoid the danger of striking the retina, zonules, or lens. In some instances, the actuator provides for a velocity of introduction of the injector into a target tissue delivery site that ranges from 1 to 10 mm/sec, such as 3.5 to 7 mm/sec. The active agent delivery system actuator may be configured to provide for control of release of an active agent composition from the therapeutic agent reservoir. The active agent delivery system actuator may be configured to provide for controlled removal of the tissue injector from the target tissue delivery site. In some such instances, the actuator may be configured to withdraw the injector from a target tissue delivery site at a velocity ranging from 1 to 10 mm/sec, such as 3.5 to 7 mm/sec. In some instances, the active agent delivery system actuator is configured to prime the tissue injector, e.g., where the therapeutic agent delivery system includes an amount of gas, e.g., air (such as in the form of bubbles) and the actuator removes the gas from the system, e.g., by causing the gas to evacuate from the system via the injector.

The active agent delivery system actuator may vary as desired. Examples of active agent delivery system actuators that may be employed in embodiments of the invention and present in the actuator component include, but are not limited to: motorized actuators (including those that include a micro-motor), as well as non-motorized actuators, e.g., pneumatic powered actuators, hydraulically power actuators, spring-loaded actuators, manually operated actuators, e.g., plunger comprising actuators, and the like. The functionality of the active agent delivery system actuator may be controlled by one or more modules, as desired.

In addition to the active agent delivery system actuator, the actuator component may include one or more components of, including all of, a pain mitigation system, e.g., as described above. Specific cooling systems of interest that may find in anesthesia producing pain mitigation systems may vary, where cooling systems of interest include, but are not limited to: thermoelectric cooling systems, liquid evaporation cooing systems, Joule-Thompson cooling systems, thermodynamic cycle cooling systems, endothermic reaction cooling systems and a low-temperature substance cooling systems. The pain mitigation system may or may not include a tissue engager, such as a cool tip, e.g., depending on whether the tissue engager is integrated with the active, e.g., therapeutic, agent delivery component, e.g., as described above.

In some instances, the pain mitigation anesthesia producing system is a thermoelectric cooling system, e.g., one that includes one or a combination of thermoelectric (Peltier) devices. While thermoelectric cooling systems employed in embodiments of devices of the invention may vary, in some instances the thermoelectric cooling systems include a cold tip that is configured to contact a target tissue delivery site (and therefore may also be referred to as a tissue engager), a power source, a controller, a cooling power concentrator, one or more Peltier unit modules, and a heat sink. It should be understood that, in some embodiments, a given thermoelectric cooling system may include a heating element (not shown) that operates in conjunction with the cooling elements to precisely maintain a desired temperature and/or heat flux. It should be understood that, in various embodiments, these elements may reside in the actuator component, the active agent delivery component, or, in some embodiments, elements reside in both the actuator component and the active agent delivery component.

In some embodiments, the cold tip (i.e., tissue engager) is made of a thermally conductive material (i.e., it includes a thermally conductive member), such as a metal (where metals of interest include, but are not limited to copper, gold, zinc, aluminum and the like), and can be sized to be generally equal to or smaller than the target tissue delivery site (e.g., the area of the ocular or other biologic surface to which the therapeutic agent is to be delivered). In some embodiments, the end of the tissue engager is circular, having a diameter ranging from 1 to 10 mm, such as 2 to 8 mm, or about 5 mm. Where desired, a thermally insulating outer ring member (e.g., that corresponds to the target area to be cooled) may be included. When present, the thermally insulating outer ring member restricts the area being cooled within the target area, which is touched by the thermally conductive cold tip, preventing damage to adjacent cells outside the target area. The cold tip may have any convenient shape, including but not limited to cylindrical, polygonal, oval, crescent, or any other conducive shape. It is noted that the tissue engager of the pain mitigation system may be integral with the other parts of the cooling system, or may be detachable from the other parts of the cooling system of the actuator, such that it is release-ably engaged to the actuator and pain mitigation system thereof, where release-ably engaged is as describe above.

In some embodiments, the power source of the pain mitigation system includes a portable power source, such as a battery, capacitor, or similar device. In some embodiments, the power source includes a rechargeable lithium ion battery pack (e.g., 28 Wh), which provides sufficient energy on a single charge to operate the device for a sufficient period of time, e.g., 0.5 to 2.5 hours, such as 1 hour. In some embodiments, the power source can include a non-portable power source.

The controller may include a temperature regulating feedback loop to maintain highly accurate temperature control and/or a timed lockout mechanism to prevent excessive cooling. In some embodiments, the controller can include a temperature sensor operably coupled with at least one member of a thermal circuit comprising the cold tip, a cooling power concentrator, one or more Peltier unit modules, a heat sink, the surrounding environment, and the target tissue delivery site to output a temperature signal in response to a detected temperature. In this way, the controller receives the temperature signal and is operable to control an operating temperature of Peltier unit module(s) via controlled current flow, controlled voltage, and/or pulse width modulation (PWM) of a power source, e.g., a DC battery source, thereby precisely regulating an operating temperature of thermoelectric cooling system. In some embodiments, the temperature sensor is arranged to directly measure the temperature of the target tissue delivery site or any portion of the thermal circuit using any one or a number of thermal sensors, such as but not limited to thermistors, thermocouples, and resistance or tissue thermometers. The controller can then compute temperature and/or heat flux. In some instances, the controller is configured to maintain a predetermined temperature or temperature range using a constant value, a pulse of certain magnitude and duration, or a more complex prescribed pattern. In some embodiments, the controller is configured to automatically power off if the tissue engager temperature falls below a certain temperature (e.g., −40° C., −35° C., −30° C., −25° C., −20° C., −15° C., −10° C., −5° C.) to ensure a safe operating temperature range, and/or if a battery temperature exceeds a certain temperature, e.g., 70° C. or the heat sink temperature exceeds a certain temperature, e.g., 140° C. In some embodiments, controller can operate on the basis of applied, measured, or desired heat fluxes rather than applied, measured, or desired temperatures.

Thermoelectric cooling systems finding use in embodiments of the invention may include a cooling power concentrator. In some embodiments, the cooling power concentrator may include an elongated concentrator made of a thermally-conductive material, such as but not limited to metal, e.g., as described above. The cooling power concentrator can be disposed along a central longitudinal axis of the actuator, and may collect cooling power from one or multiple Peltier units. In some embodiments, the cooling power concentrator can be polyhedron in shape, and the cooling power collected from the surface(s) in contact with Peltier unit(s) is concentrated to one or more surfaces whose aggregate area is less than that of the Peltier unit cooling surface(s) at which collection occurs. However, it should be understood that the cooling power concentrator can have other shapes, including cylinder, cone, conical cylinder, sphere, hemisphere, or any other shapes that provide collecting and concentrating of cooling power. In such embodiments, the Peltier unit module(s) can be shaped to define a complementary surface to enhance surface area contact between Peltier unit module(s) and the cooling power concentrator to facilitate thermoelectric cooling.

In addition to the above component, a thermoelectric cooling system may include a heat sink. In some instances, a heat sink is made of a thermally conductive material to efficiently spread the heat rejected from Peltier unit module(s) of the system. In some embodiments, a heat sink is radially disposed about cooling power concentrator and Peltier unit module(s). In other words, a heat sink radiates outwardly from a central longitudinal axis of the actuator component. However, it should be understood that a heat sink can radiate heat in other directions depending on the relative angle of the hot surface of Peltier unit module(s) with respect to central cooling portion of cold tip.

Further details regarding embodiments of thermoelectric cooling systems that may be employed in devices of the invention are provided in U.S. Published Patent Application Publication No. 20160279350; the disclosure of which is herein incorporated by reference.

As indicated above, in some instances, the actuator component may include a component of a locking element for release-ably engaging the active agent delivery component to the distal end of the actuator component of the device. As reviewed above, any convenient locking mechanism may be employed, such as but not limited to: luer-lock, screw on, snap on, and the like. The locking element component of the locking element that is present on the actuator component may vary, as desired, and is selected based on the companion element that is present on the actuator.

In some instances, the actuator component includes an identifier reader for reading an identifier of an active agent delivery component release-ably engaged with the actuator component. The identifier reader may vary, as desired, depending on the nature of the identifier that is associated with the active, e.g., therapeutic, agent delivery component. For example, where the identifier is a barcode, the identifier reader of the actuator may be any convenient barcode or QR code scanner. Likewise, where the identifier is a radiofrequency identifier, the identifier reader of the actuator may be any convenient RFID reader, and together with the RFID chip, may comprise either a passive RFID or active RFID system. The identifier reader, when present, is located on the actuator at a position such that it is reading relationship with the identifier, and can therefore functionally interact with the identifier, of the active agent delivery component when release-ably engaged with the actuator. In some instances, the actuator component may be configured to read one or more additional identifiers that are not associated with an active agent delivery component, where in such embodiments the actuator component may include a separate identifier reader for such non-active agent delivery component identifiers, or an identifier reader that can read identifiers of the active agent delivery component as well as other identifiers, e.g., as described in greater detail below.

Where the actuator component includes an identifier reader, in some instances the actuator is configured to be active only when the identifier reader detects an acceptable identifier. An acceptable identifier may be an identifier that imparts one or more types of information upon which acceptability may be based, such as but not limited to: whether the active agent delivery component is filled with the correct active, e.g., therapeutic, agent; whether the active agent delivery component indicates the correct active, e.g., therapeutic, agent; whether the active, e.g., therapeutic, agent delivery component is expired; whether the active, e.g., therapeutic, agent delivery component is manufactured by an acceptable, authentic source; whether the active, e.g., therapeutic, agent delivery component has been previously registered as lost; whether the active, e.g., therapeutic, agent delivery component is intended for a correct patient, etc. In such instances, the reader may be coupled to an actuator control element that only enables one or more actuator components, such as the active agent delivery system actuator, the pain mitigation system, etc., when an acceptable identifier is read by the identifier reader. As such, where an unacceptable identifier is read by the reader, the reader may send a single to the controller that disables one or more of the actuator components. Alternatively, where an unacceptable identifier is read by the reader, the reader may send a signal to the controller that one or more of the actuator components should not be enabled.

In some instances, the actuator component may include an active agent delivery component determination module, which module is configured to obtain a data from the identifier via the identifier reader and make an active agent delivery component determination based thereon, such as whether the active agent delivery component is acceptable for a given dosage administration protocol. As defined above, modules are made up of one or more functional blocks which act in concert to perform a particular function, which particular function is the purpose of the module. A given module may be implemented as hardware, software or a combination thereof. In some instances, modules may include a circuitry element, such as an integrated circuit. When present, integrated circuits may include a number of distinct functional blocks, where the functional blocks are all present in a single integrated circuit on an intraluminal-sized support. By single integrated circuit is meant a single circuit structure that includes all of the different functional blocks. As such, the integrated circuit is a monolithic integrated circuit (also known as IC, microcircuit, microchip, silicon chip, computer chip or chip) that is a miniaturized electronic circuit (which may include semiconductor devices, as well as passive components) that has been manufactured in the surface of a thin substrate of semiconductor material. In addition to a determination as to whether a given active, e.g., therapeutic, agent delivery component is acceptable, the active, e.g., therapeutic, agent delivery component determination module of the actuator component may be configured to make or more additional types of active, e.g., therapeutic, agent delivery component determinations, including but not limited to, those described in greater detail below.

Actuator components of the invention may further include a communications module, which module is operably coupled to one or more components of the actuator and provides for data transfer therefrom to another component, e.g., an external device, etc. The communications module may be configured to provide for the transfer of data in a wired or wireless mode, as desired. For example, the communications module may be configured to transfer data, e.g., with a networked device, while being used, and/or when docked at a docking station, such as described below. The communication may be wireless, or, when connected to the docking station, either wired or wireless. Communications modules of the actuators may be configured, e.g., via hardware and/or software implementation, to perform desired communications functions, e.g., to receive data from an actuator element, to transfer data, e.g., to a USB port for wired communications or a wireless transmitter for wireless communications, etc. Communications modules (as well as any other modules described herein, such as actuator controller modules, etc.) are made up of one or more functional blocks which act in concert to perform a particular function, which is the purpose of the module. A given communications module may be implemented as hardware, software or a combination thereof. In some instances, the communications module may include a circuitry element, such as an integrated circuit. When present, integrated circuits may include a number of distinct functional blocks, i.e., modules, where the functional blocks are all present in a single integrated circuit on an intraluminal-sized support. By single integrated circuit is meant a single circuit structure that includes all of the different functional blocks. As such, the integrated circuit is a monolithic integrated circuit (also known as IC, microcircuit, microchip, silicon chip, computer chip or chip) that is a miniaturized electronic circuit (which may include semiconductor devices, as well as passive components) that has been manufactured in the surface of a thin substrate of semiconductor material.

Where desired, actuator components may include a variety of different types of power sources that provide operating power to the actuator component in some manner. The nature of the power source may vary, and may or may not include power management circuitry. In some instances, the power source may include a battery. When present, the battery may be a onetime use battery or a rechargeable battery. For rechargeable batteries, the battery may be recharged using any convenient protocol. In some applications, the actuator may have a battery life ranging from 0.1 to 14 hrs, such as 0.5 to 10 hrs or 1 hour to 5 hours.

In certain instances, the actuator of the invention includes an updatable control module, by which is meant that the actuator is configured so that one or more control algorithms of the actuator may be updated. Updating may be achieved using any convenient protocol, such as transmitting updated algorithm data to the control module using a wire connection (e.g., via a USB port on the device) or a wireless communication protocol. The content of the update may vary. In some instances, an actuator component is updated to configure the unit to be used with a particular active, e.g., therapeutic agent delivery component. In this fashion, the same actuator component may be employed with two or more different active, e.g., therapeutic, agent delivery components that may differ by from each other in one more ways, e.g., identify of therapeutic agent, manufacturer of therapeutic agent delivery component, etc. The update information may also include general functional updates, such that the actuator component can be updated at any desired time to include one or more additional software features and/or modify one or more existing programs of the device. The update information can be provided from any source, e.g., a particular elongated member, the internet, etc.

The actuator component may include one or more safety mechanisms, e.g., in addition to or instead of, the identifier/reader compatibility mechanism as described above. In some embodiments, the active, e.g., therapeutic, agent delivery system actuator will provide for actuation only if a switch is depressed continuously during the injection process. In some embodiments, there will be a safety mechanism to halt injection.

As indicated above, in some instances, the actuator component may include a component of a locking element for release-ably engaging the active agent delivery component to the distal end of the actuator component of the device. As reviewed above, any convenient locking mechanism may be employed, such as but not limited to: luer-lock, screw on, snap on, and the like. The locking element component of the locking element that is present on the actuator component may vary, as desired, and is selected based on the companion element that is present on the actuator.

The actuator components of the invention may be fabricated using any convenient materials or combination thereof, including but not limited to: metallic materials such as tungsten, stainless steel alloys, platinum or its alloys, titanium or its alloys, molybdenum or its alloys, and nickel or its alloys, etc.; polymeric materials, such as polytetrafluoroethylene, polyimide, PEEK, and the like; ceramics, such as alumina (e.g., STEATITE™ alumina, MAECOR™ alumina), etc.

In some instances, the actuator components may include a display. By display is meant a visual display unit, which may include a screen that displays visual data in the form of images, lights, and/or text to a user. The screen may vary, where a screen type of interest is an LED screen or LED-backlit LCD screen. The display, when present, may be integrated with the actuator component. As such, the display may be an integrated structure with the actuator component, such that it cannot be separated from the actuator component without damaging the monitor in some manner. The display, when present will have dimensions sufficient for use with the actuator, where screen sizes of interest may include 100 cm² or smaller, such as 20 cm² or smaller, etc. The display may be configured to display a variety of different types of information to a user, where such information may include devices settings, including tip temperature, time of cooling application, active, e.g., therapeutic, agent identification, and active, e.g., therapeutic, agent expiration date, etc.

### Smart Device Configurations

As described above, in some instances the active, e.g., therapeutic, agent delivery component includes an identifier and the actuator component includes an identifier reader, such that the device may be viewed as a “smart” device. In such embodiments, a variety of different types of information may be stored on the identifier. Reading of the identifier by the identifier reader, e.g., when the active agent delivery component is release-ably engaged to the actuator component, transmits information to the actuator component. Reading of the identifier by the identifier reader also stores information regarding successful or unsuccessful delivery of the active, e.g., therapeutic, agent. This stored information can be later transmitted, e.g., via wired or wireless communication, to a separate device, such as a networked device, e.g., in the form of a computer or server. Cumulative use data, including, but not limited to, the number of injections of a specific agent, the mean number of days drugs were injected prior to their expiration date, the number of rejected drug cartridges and the like, can be accrued by the identifier reader and stored for subsequent transfer to another device, such as a networked device, e.g., via download or wireless transmission.

In some instances, the identifier includes active agent delivery component information. Active agent delivery component information is information or data about the active agent delivery component itself. Such information may include active agent delivery component historical information. Historical information is information about the nature of the active agent delivery component and/or one or more past events experienced by the active agent delivery component. Historical information includes, but is not limited to: an active agent identifier (e.g., the name of the active agent (or a proxy thereof) contained in the active agent delivery component); manufacturing information, such as manufacturing lot number and/or source for an active agent and/or active agent delivery component; active agent delivery component handling information (e.g., information about the supply channel through which the active agent delivery component has passed, e.g., chain of custody information, may include environmental condition information (e.g., shipment tracking information including time and geographical information, and temperature information over time, such as any storage temperature excursions that may have occurred, etc.)); active agent delivery component dose, concentration, and/or volume information; active agent delivery component expiration date information; and the like. As such, historical information may include information about a particular active, e.g., therapeutic, agent contained in the active agent delivery component. Such information may include, but is not limited to: identity of the active, e.g., therapeutic, agent (brand name and/or generic name); date of manufacture; lot number; date of expiry; source of manufacture; handling and storage information; dosage amount; delivery volume; drug concentration; intended route of administration; identity of patient information; indication for use; whether the component has ever been reported lost or stolen; whether the component has ever been previously used; etc.

In some instances, the identifier includes active agent delivery component information that is active agent delivery component use information. Active agent delivery component use information is information or data about the actual use of the active agent delivery component, e.g., the actual employment of that active agent delivery component to deliver an active, e.g., therapeutic, agent to a target delivery site. Such information may vary, and may include, but is not limited to: use date information (i.e., information about the date, time, etc., at which the component was used); administration information, (e.g., confirmation that actual delivery to a subject occurred, identity of the subject to which the active, e.g., therapeutic, agent was administered, condition of the subject for which the active agent was administered, and the like); etc.

In some instances, the identifier includes information that can be used following administration of the agent. This information may include the ability to re-order a delivery component after it has been used, generate or transfer information that could be helpful for submission to insurance for reimbursement, generating a new appointment or appointment request for a patient, initiate a payment to the supplier for use of the agent, or notate the patient's electronic medical record (EMR). Additionally, the identifier could include information to initiate a service request, for example, if the actuator did not successfully administer the agent, or if the device is due for routine maintenance.

In some instances, the active delivery component information includes a linked sensor, e.g., as described above. In such instances, an identifier may include information obtained from the sensor. Sensor-derived information can then be used in a variety of different applications, e.g., to help determine whether the therapeutic agent is suitable for administration. For example, if the active agent has not been stored at the proper temperature, the actuator component could dis-qualify, rather than deliver, the active, e.g., therapeutic, agent, and trigger an order to replace the spoiled active agent.

As described above, in some instances, the actuator component may be configured to read one or more additional identifiers that are not associated with a active agent delivery component, where in such embodiments the actuator component may include a separate identifier reader for such non-active agent delivery component identifiers, or an identifier reader that can read identifiers of both the active agent delivery component as well as other, non-active agent delivery component identifiers. An actuator may be configured to read a variety of different non-active agent delivery component identifiers, where examples of such identifiers include, but are not limited to: patient associated identifiers, where such patient associated identifiers may be physically associated with a patient (e.g., on a wristband), associated with a patient's medical record, such as an electronic medical record (EMR), etc.; operator associated identifiers, where such operator associated identifiers may be physically associated with an operator of the device (such as present on a badge or other operator associated identification device), associated with an operator's personnel records, etc; and the like. Non-active agent delivery component identifiers that may be read by an actuator component may vary, e.g., as described above, and included but are not limited to: barcodes, RFID tags, NFC tags, etc.

In those embodiments where an actuator is configured to read non-active agent delivery component identifiers, the actuator may be configured to modulated in a variety of different ways based on information received by the actuator from such identifiers. For example, a device may be configured such that an actuator reads identifiers from both an active agent delivery component and a patient, and actuates the delivery device only when information received from the identifiers matches, e.g., as determined by an algorithm, which may be present on the actuator or on a networked device. For example, in some instances a device is configured so that it can only be activated if the following occur: 1) the actuator scans an identifier, such as a barcode/RFID tag/NFC tag, associated with a patient (e.g., present on a wristband); the actuator scans an identifier, such as a barcode/RFID/NFC tag, affixed to the actove agent delivery component; and 3) information received from the identifiers is determined to match, e.g., the patient is one that has been prescribed the active agent present in the active agent delivery component, such that the device is activated and is able to deliver the active agent to the patient. In those situations where the information from the identifiers is determined not to match, e.g., information obtained from the patient identifier states that an active agent different from that in the active agent delivery component is to be administered to a patient, the device is not activated and is unable to deliver the active agent to the patient.

In some instances, the actuator component actively communicates with an auto-generated barcode/RFID/NFC tag generated inside the patient's electronic medical record (EMR) after a drug specific order has been placed. Device activation in such embodiments includes communication with a physically associated patient identifier; a patient EMR associated identifier and the active agent delivery component identifier.

In some instances, activating the device through scanning a patient barcode and scanning/affixing the active, e.g., therapeutic, agent delivery component results in one or more of: an order being placed in the EMR, auto-generates a procedure note, generates a bill, triggers a follow-up appointment, etc.

In some instances, the device activator may be programmed to recognize and utilize artificial intelligence data to help ensure selection of an ideal active, e.g., therapeutic, agent for a given patient based on predetermined criteria. In one non-limiting example, the device activator may be configured to obtain patient-specific data related to central macular thickness changes following treatment with an active, e.g., therapeutic, agent from a linked optical coherence tomography machine. This data transmission could then be read by the device activator, resulting in a specific therapeutic agent being selected for delivery. Without direct override, no other active, e.g., therapeutic, agents would be recognized as appropriate for that specific patient.

As will be appreciated by the skilled artisan, this information could be stored directly on the identifier, or could be looked up in a linked database using the identifier information, e.g., as may be presented on a networked device, such as described in greater detail below.

Smart device embodiments, e.g., as described above, allow for one or more desirable capabilities, including but not limited to inventory management capabilities, enhanced therapeutic capabilities, medical record history capabilities, data analytics capabilities, and the like. For example, a variety of different inventory management capabilities are provided by smart device embodiments, including automated reordering of therapeutic agent delivery component by a user (e.g., according to user preset preferences), tracking of individual therapeutic agent delivery components (e.g., to manage lost, stolen, or expired goods components), and the like. A variety of different enhanced therapeutic capabilities are provided by smart device embodiments, including auto-generation of procedure notes, communication with existing electronic medical records for integration in a patient chart, sending data on drug/dose/route for documentation purposes and/or billing purposes, aggregating data on drug/dose/route for market research and analytics, facilitating documentation for reimbursement, and the like. Examples of capabilities provided by smart device embodiments, e.g., as described herein, are further provided in Published United States Patent Application Publication Nos. 20160030683; 20170098058; 20170119969; 20170124284 and 20170124285; the disclosures of which capabilities are incorporated herein by references.

### Specific Embodiments

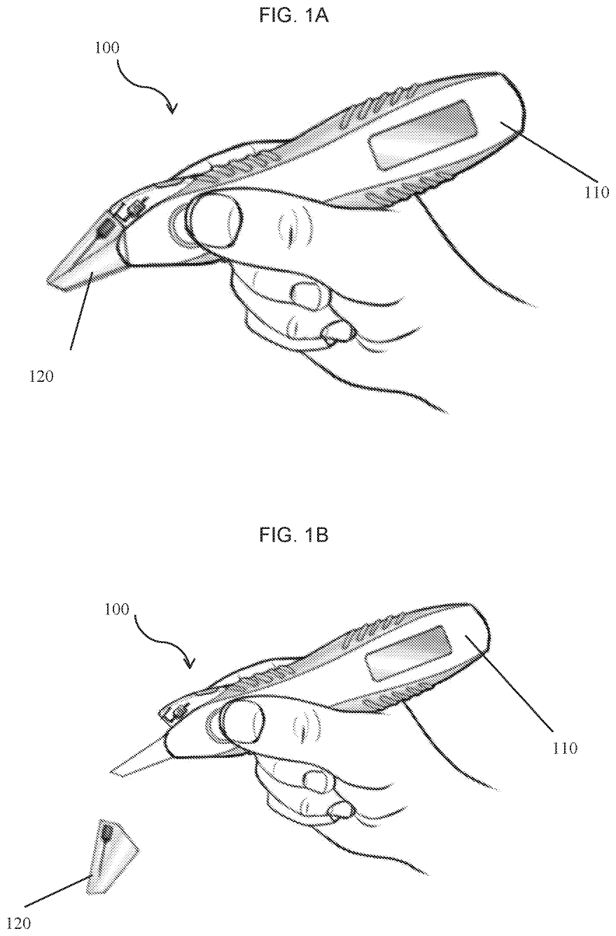

FIG. 1A provides a view of a device according to an embodiment of the invention that is configured to deliver a therapeutic agent to an ocular target delivery site. As shown in FIG. 1A, device 100 is a handheld device. The device includes an actuator component 110 and a therapeutic agent delivery component 120. As illustrated in FIG. 1A, the therapeutic agent delivery component 120 is release-ably engaged to the distal end of the actuator component. FIG. 1B provides a view of the same device in disassembled form, where therapeutic agent delivery component 120 is separated from the actuator component 110.

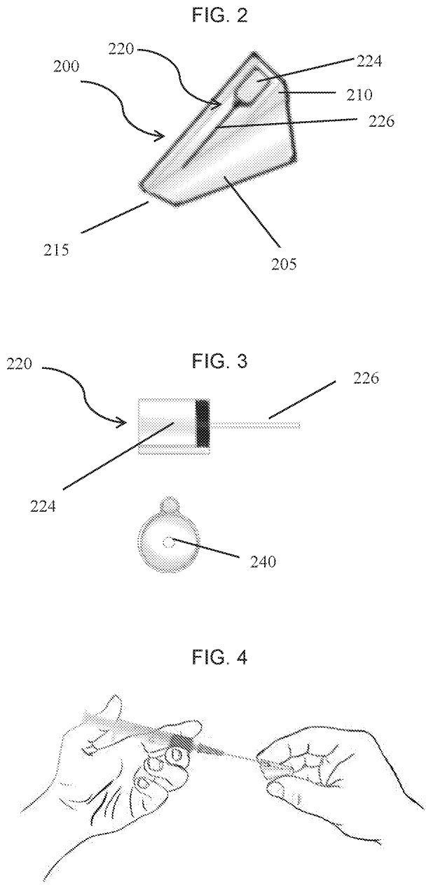

FIG. 2 provides a view of a therapeutic agent delivery component according to an embodiment of the invention. As shown in FIG. 2, therapeutic agent delivery component 200 includes a body 205 having a proximal end 210 that is configured to be release-ably engaged to the distal end of an actuator component, such as actuator component 110 of FIGS. 1A and 1B, and distal end 215 that is configured to contacted to an ocular tissue delivery site during therapeutic agent delivery. Also shown is therapeutic agent delivery system 220 that includes reservoir 224 and needle 226. Finally, therapeutic agent delivery component 200 includes a passage 230 configured to accommodate a cold tip of a cooling system of an actuator component, e.g., such as actuator component 110 of FIGS. 1A and 1B.

FIG. 3 provides side and back views of the therapeutic agent delivery system 220. As shown in FIG. 3, therapeutic agent delivery system 220, includes reservoir (or ampoule) 224 and needle 226. Also shown is port 240 located at one end of reservoir 224 which allows for filling of the reservoir with a therapeutic agent prior to use, e.g., as illustrated in FIG. 4. While FIG. 4 shows the loading of the reservoir with a therapeutic agent composition, in some instances a therapeutic agent delivery component is provide by a manufacturer prefilled with a therapeutic agent composition in the reservoir.

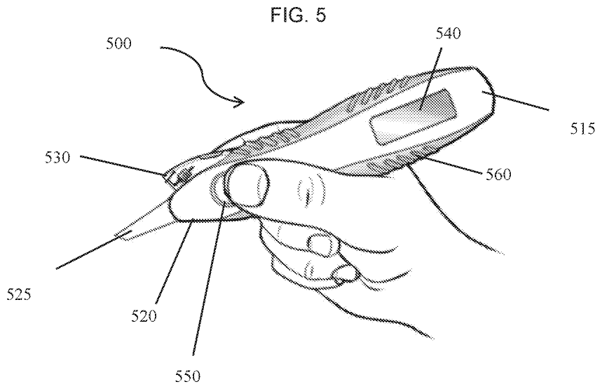

FIG. 5 provides a view of an actuator component according to an embodiment of the invention. As shown in FIG. 5, actuator component 500 includes a proximal end 510 and a distal end 520. Also shown is cold tip 525 of a thermoelectric cooling pain mitigation system of the actuator. The cold tip 525 may be a cold tip such as described above, and may be part of a cooling system e.g., as described in U.S. Published Patent Application Publication No. 20160279350; the disclosure of which is herein incorporated by reference. Actuator component 510 further includes a therapeutic delivery system actuator 530, which system includes a micro-motor that operates to move a needle forward from a therapeutic agent delivery system in a release-ably engagement therapeutic agent delivery component, where the needle is moved forward into a target tissue delivery site. In some instances, the actuator moves the needle forward at a speed ranging from 3.5 to 7 mm/sec, where the speed may be constant or varied as desired. The actuator 530 also controls withdrawal of the needle from the ocular tissue delivery site following delivery of the therapeutic agent. In some instances, the actuator moves the needle backward at a speed ranging from 3.5 to 7 mm/sec, where the speed may be constant or varied as desired. Actuator component 500 also includes a display 540, an activator button 550 and manual grips 560.

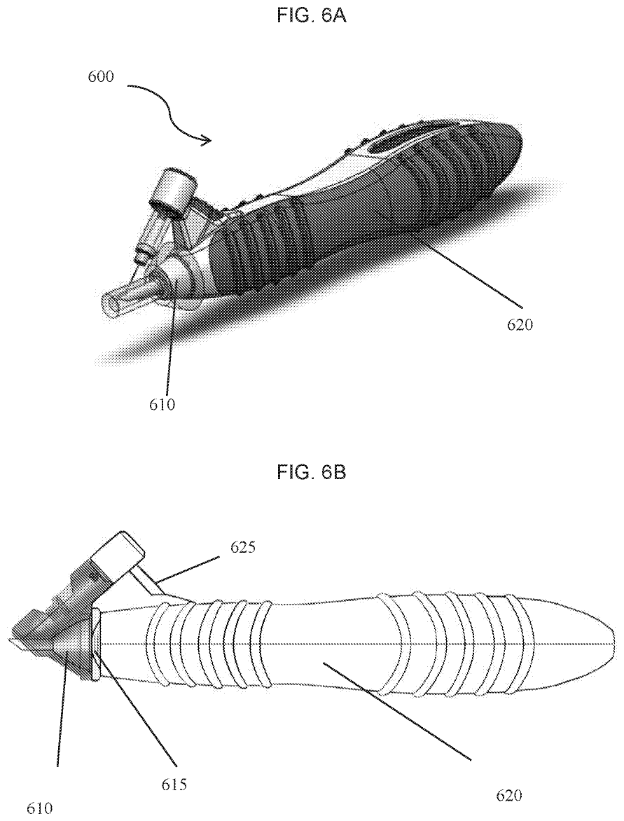

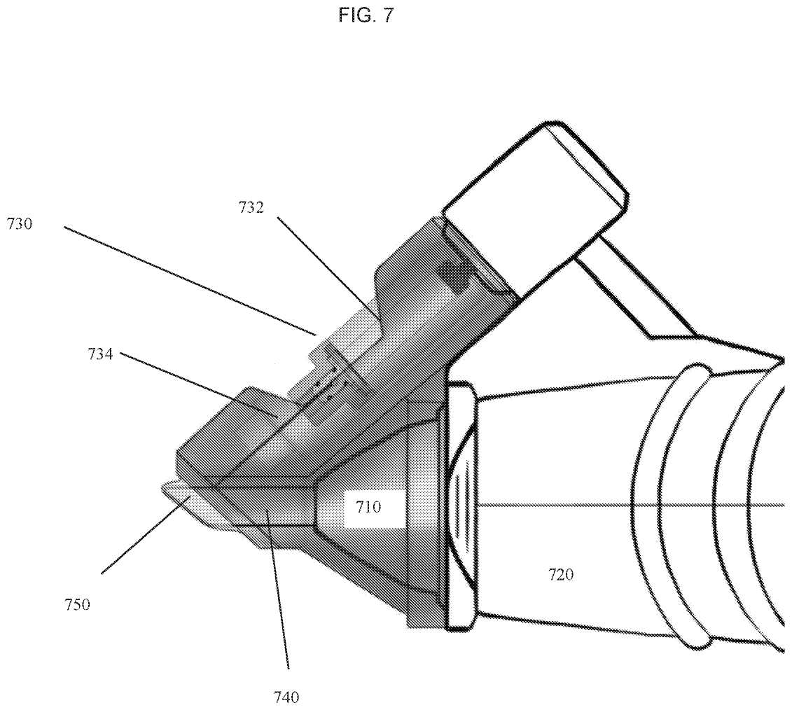

FIG. 6A provides a perspective view of a device 600 according to another embodiment of the invention. As shown in FIG. 6A, device 600 includes a therapeutic agent delivery component 610 and an actuator component 620. FIG. 6B provides a side view of the device 600 shown in FIG. 6A. Also shown in FIG. 6B cartridge ejector button 615 which may be used to separate a used therapeutic agent delivery component 610 from the actuator 620, and a manual therapeutic agent delivery system button 625 which may be depressed to move a needle of the therapeutic agent delivery system of the therapeutic agent delivery component into an ocular delivery site during use.

FIG. 7 provides a side view of a therapeutic agent delivery component 710 release-ably engaged to a distal end of an actuator component 720. As shown in FIG. 7, therapeutic agent delivery component 710 includes a therapeutic agent delivery system 730 that includes a reservoir or ampoule 732 and a needle 734. Also shown is cool tip passageway 740 and a thin membrane 750 at the distal end of therapeutic agent delivery component, which membrane 750 provides for heat transfer between the cool tip and the target tissue delivery site, e.g., as described above.

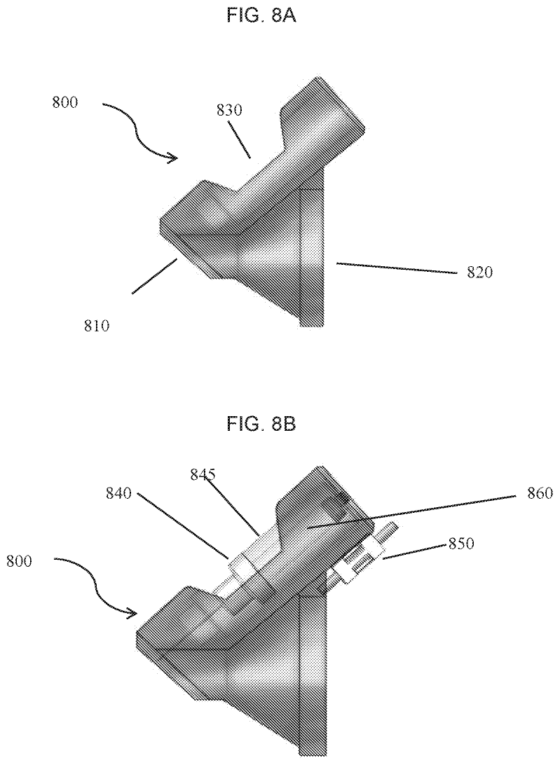

FIGS. 8A to 8B provide views of a different sub-components of a therapeutic agent delivery component according to an embodiment of the invention. Shown in FIG. 8A is a therapeutic agent delivery component body 800 having a proximal, actuator component engaging end 820 and a distal, target tissue engaging end, 810. Also shown is space 830 for a therapeutic agent delivery system. FIG. 8B shows the body 800 of FIG. 8A having a therapeutic agent delivery system 840 loaded therein. As seen, therapeutic agent delivery system 840 includes a viewing window 845 that provides for viewing of an ampoule 860 of the therapeutic agent delivery system. Also shown in micro-motor 850 for actuating the therapeutic agent delivery system 840.

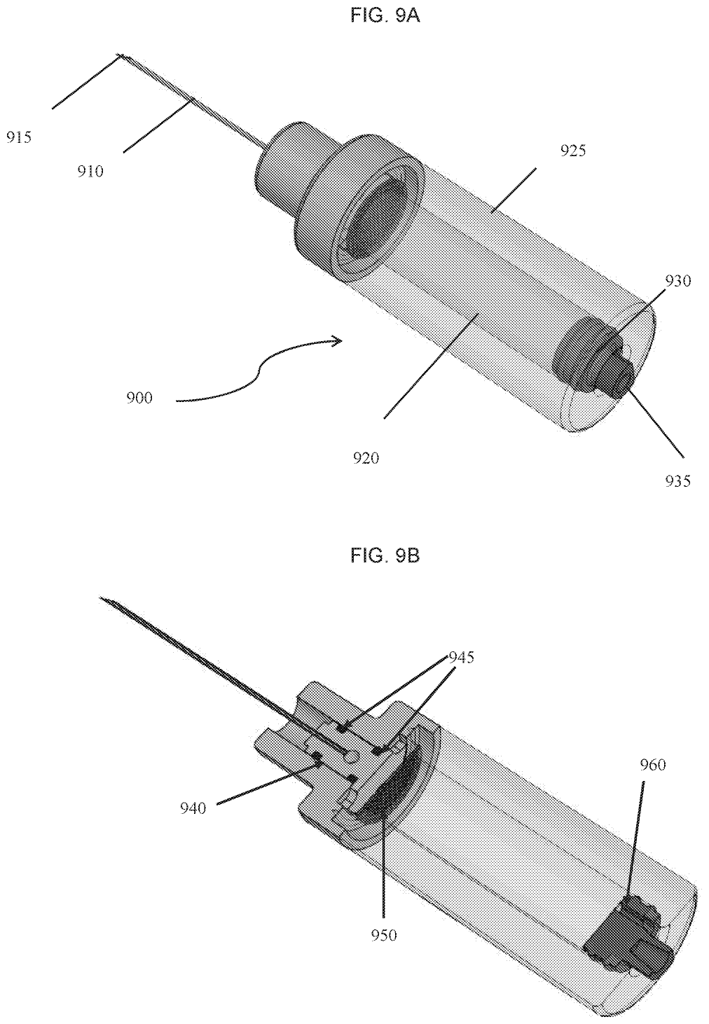

FIGS. 9A and 9B provide views of a therapeutic agent delivery system according to an embodiment of the invention. As shown in FIG. 9A, therapeutic agent delivery system 900 includes a needle 910 having a beveled tip 915 fluidically coupled to reservoir or ampoule 920, which is made of a transparent material, e.g., glass. Ampoule 920 has a plunger 930 at its distal end that is configured to be operably engaged to a therapeutic agent delivery system actuator of an actuator component. Plunger 930 includes filling port 935. Ampoule 920 is housed in an optically transparent cylindrical housing 925 that provides for a viewing window when the therapeutic agent delivery system is present in a body of a therapeutic agent delivery component. FIG. 9B provides a cutaway view of the therapeutic agent delivery system shown in FIG. 9A. As shown in FIG. 9B, therapeutic agent delivery system 900 further includes a needle hub 940 having a pair of O-rings 945 that fluidically connects the needle to the ampoule, and a filter 950 positioned at the distal end of the ampoule. Also shown is venting port 960 which is present on the plunger.

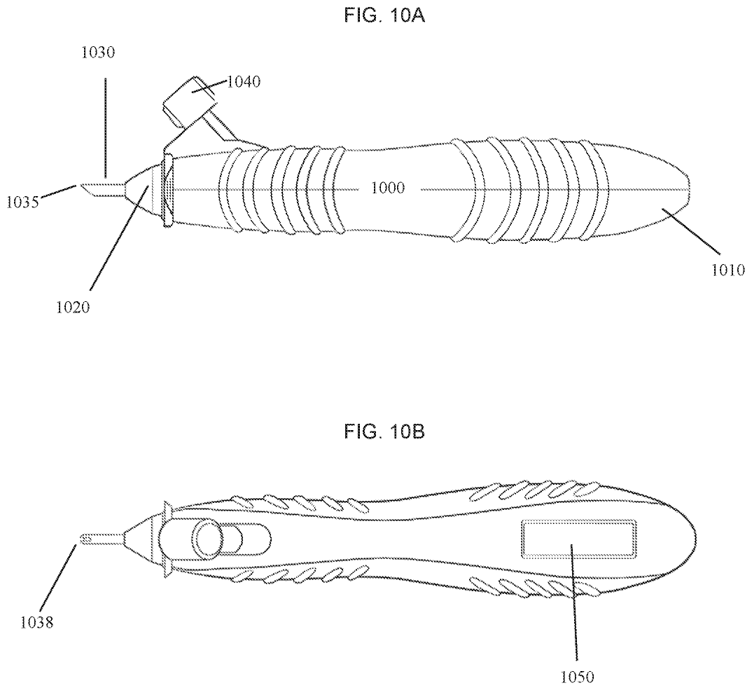

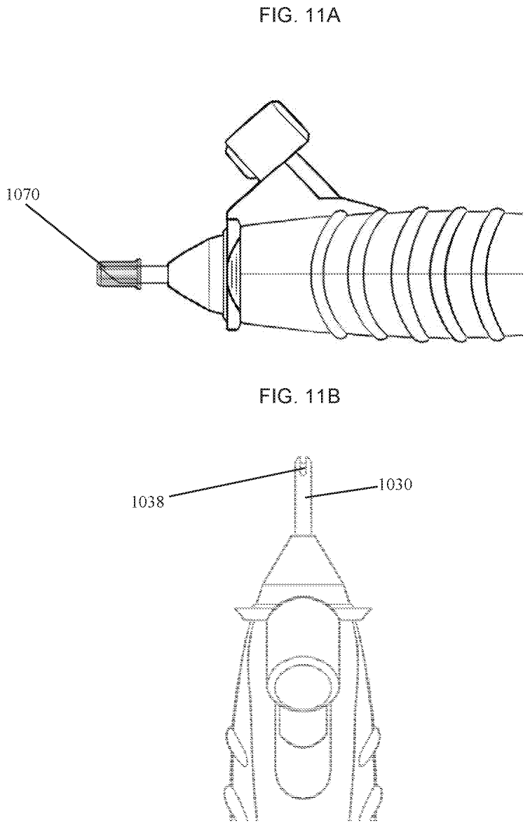

FIGS. 10A and 10B provide views of an actuator component according to an embodiment of the invention. FIG. 10A provides a side view of an actuator component. As shown in FIG. 10A, actuator 1000 includes proximal end 1010 and distal end 1020. Located at distal end 1020 is cool tip 1030 which has a beveled end 1035 which is configured to provide for controlled needle injection during use, e.g., as described in greater detail below. Also shown is therapeutic agent delivery system actuator 1040. FIG. 10B provides a top view of the actuator component 1000 shown in FIG. 10A. As shown in FIG. 10B, actuator component 1000 includes a display 1050. In addition, cool tip 1030 includes a needle guide 1038 which provides for a controlled angle of needle injection during use.

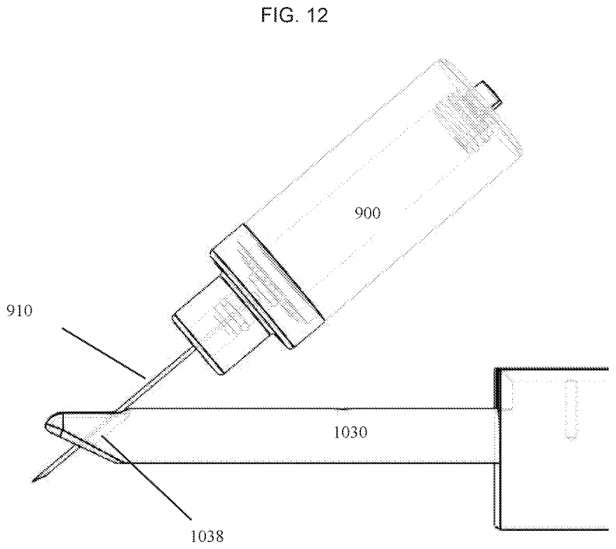

FIGS. 11A and 11B provide close up views of the distal end of the actuator component 1000 of FIGS. 10A and 10B. In FIG. 11A, prior to use an actuator component 1000 may include a removable, sterile cover 1070 that maintains sterility of the tissue engaging surfaces of cool tip 1030. FIG. 11B provides a overhead view of the cool tip 1030 and needle guide 1038 which provides for a controlled angle of needle injection during use.

FIG. 12 provides a view of a needle 910 of a therapeutic delivery system 900 engaged in the needle guide 1038 of cool tip 1030 of an actuator component. As shown in FIG. 12, needle guide 1038 and beveled end 1035 of cool tip 1030 are configured such that the need extends at a defined angle into a target tissue delivery system, such as an ocular tissue delivery site, when the beveled end 1035 is placed flush against the target tissue delivery site. As the extent to which needle 910 extends beyond the beveled tip 1035 is controlled by the actuator, highly controlled and reproducible therapeutic agent delivery is achieve, which may provide for a number of advantages, including enhanced safety, enhanced accuracy of delivery, etc., resulting in improved therapeutic outcomes.

### Systems

Aspects of the invention further include systems that include a device of the invention, e.g., as described above, or components thereof, in communication with one or more networked devices. As such, systems of the invention may include an active agent delivery component and/or an actuator component in communication with a networked device.

A networked device is any device that communicates with at least one other device over a communication link, and in the present invention is a device that includes a communications module that is configured to communicate with the communications module of the active, e.g., therapeutic, agent delivery device, either directly or via one or more intermediate devices. Networked devices that may be part of a system of the invention may vary, where such devices include, but are not limited to: desktop computing devices, intermediate computing devices, mobile devices (e.g., laptop, cell phone or other mobile computing devices), servers (which may be local or remote), etc. The communication link may vary, where the communication link may be a wired or wireless communication link. Wired communication links may include USB, FireWire, HDMI, Ethernet, LAN, and the like. Wireless communication links that may be employed include, but are not limited to, those employed in any suitable communications network, such as but not limited to wireless personal area networks (WPANs) (e.g., Bluetooth, ZigBee), wireless local area networks (WLANs) (WiFi), wireless ad hoc networks, wireless metropolitan area networks, wireless wide area networks, cellular networks, global area networks, etc.

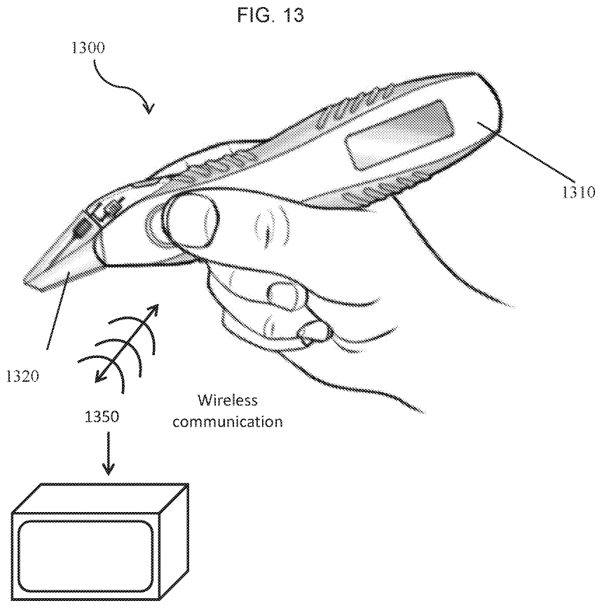

An illustration of a system is shown according to an embodiment of the invention is provided in FIG. 13. FIG. 13 provides a view of a device according to an embodiment of the invention that is configured to deliver a therapeutic agent to an ocular target delivery site. As shown in FIG. 13, device 1300 is a handheld device. The device includes an actuator component 1310 and an active agent delivery component 1320. As illustrated in FIG. 13, the active agent delivery component 1320 is release-ably engaged to the distal end of the actuator component 1310. Also shown in FIG. 13 is networked device, 1350, which device may be any of a number of different devices. In the embodiment illustrated in FIG. 13, networked device 1350 is in wireless communication with device 1300.

While the networked device may vary, in some instances a networked device includes an active agent delivery component determination module. Such a module is configured to receive data from an active agent delivery device or component thereof, e.g., an actuator component and/or an active agent delivery component, and make a determination about an active agent delivery component, e.g., as described in more detail below, from the received information. The resultant active agent delivery component determination may be used in a variety of applications, as described in greater detail below.

In some instances, active agent delivery component determination modules include: a processor coupled to a communications module and a memory coupled to the processor, where the memory stores non-transitory computer-readable instructions, e.g., in the form of software, that, when executed by the processor, cause the processor to make an active agent delivery component determination based on data received from the active agent delivery device, component(s) thereof. The nature of the active agent delivery component determination may vary depending on the particular application in which the active agent delivery device is being employed.

In some instances, the active agent delivery component determination includes active agent delivery component historical information. As reviewed above, active agent delivery component historical information may vary. In some instances, active agent delivery component historical information includes identifying information about an active agent delivery component and/or a therapeutic composition thereof, such as but not limited to: active, e.g., therapeutic, agent name; active agent dose, concentration and/or volume information; indications for use; and the like. In some instances, active agent delivery component historical information includes manufacturing information about an active agent delivery component and/or an active composition thereof, such as but not limited to: manufacturing data for an active agent delivery component and/or an active agent composition present therein (such as, but not limited to, manufacturing date, manufacturer identity, manufacturer lot number, unique vial identification number, etc.). In some instances, active agent delivery component historical information includes handling information about an active agent delivery component and/or an active agent composition thereof, such as but not limited to: handling data about the active agent delivery component and/or active agent composition thereof, such as but not limited to: supply chain information (where such information may include sterility/contamination information; temperature (or temperature history); geographical information (e.g., locations through which the active agent traveled), exposure to humidity, exposure to radiation, etc.); handler information (e.g., whether only authorized entities have handled the component); accounting information (e.g., whether the component has been identified as being lost or stolen, billing information, reimbursement information, etc.); and the like. In some instances, active agent delivery component historical information includes age information about an active agent delivery component and/or an active composition thereof, such as but not limited to expiration date, use by date, and the like.

A determination made by a networked device that includes historical information, such as described above, may be employed in a variety of different applications. For example, such a determination may be employed in an active agent delivery device control process. By active agent delivery control process is meant a protocol or workflow in which a determination is made whether or not to deliver, or what dose to deliver, the active agent to particular subject. An example of such an active agent delivery device control process is one that controls actuation of the delivery device based on an active agent delivery component determination. For example, based on the determination, the networked device may transmit a signal to a communicatively linked active agent delivery device that either enables or disables the device, or configures a given dose setting. For example, based on the determination, a signal may be transmitted that enables the device for delivery, where examples of determinations that may result in such an enabling signal include, but are not limited to, determinations that the active agent delivery component and/or active agent composition thereof is one or more of: prescribed for the subject, e.g., as may be verified by evaluating a medical record, such as an electronic medical record; is the correct dose; is genuine and non-counterfeit; has been obtained from an acceptable manufacturer; has not expired; has not experienced supply chain compromises; has not been reported lost or stolen; has not been previously used; and the like. In some instances, based on the determination, a signal may be transmitted that modulates the delivered dose. In some cases, the dose may be disabled or set to zero, where examples of determinations that may result in such a disabling signal include, but are not limited to, determinations that the active agent delivery component and/or active agent composition thereof is one or more of: not prescribed for the subject, e.g., as may be verified by evaluating a medical record, such as an electronic medical record; not obtained from an acceptable manufacturer; has expired; has experienced supply chain compromises; has been reported lost or stolen; has been previously used and the like. Enablement or disablement of an active agent delivery device may be accomplished in any convenient way. For example, a networked device may send a signal to the device that activates or inactivates the actuator component, as desired. Alternatively, the networked device may send a signal to the device that allows or prevents an active agent delivery component from being release-ably engaged to an actuator component to assemble a functional delivery device. Alternatively, the networked device may send a signal to deliver a different dose; in some cases, this dose may be zero, which is functionally equivalent to disabling the device.

As described above, in some instances, the actuator component may be configured to read one or more additional identifiers that are not associated with an active agent delivery component, where in such embodiments the actuator component may include a separate identifier reader for such non-therapeutic agent delivery component identifiers, or an identifier reader that can read identifiers of both the active agent delivery component as well as other, non-active agent delivery component identifiers. An actuator identifier reader may be configured to read a variety of different non-actuator agent delivery component identifiers, where examples of such identifiers include, but are not limited to: patient associated identifiers, where such patient associated identifiers may be physically associated with a patient (e.g., on a wristband), associated with a patient's medical record, such as an electronic medical record (EMR), etc.; operator associated identifiers, where such operator associated identifiers may be physically associated with an operator of the device (such as present on a badge or other operator associated identification device), associated with an operator's personnel records, etc.; and the like. Non-active agent delivery component identifiers that may be read by an actuator component may vary, e.g., as described above, and included but are not limited to: barcodes, RFID tags, NFC tags, etc.

In some instances, a system of the invention includes a component configured to produce one or more non-active agent delivery component identifiers, such as described above. Examples of such identifiers include, but are not limited to: patient associated identifiers, where such patient associated identifiers may be physically associated with a patient (e.g., on a wristband), associated with a patient's medical record, such as an electronic medical record (EMR), etc.; operator associated identifiers, where such operator associated identifiers may be physically associated with an operator of the device (such as present on a badge or other operator associated identification device), associated with an operator's personnel records, etc.; and the like. For example, a given system may include a printer configured to produce a barcode identifier, which identifier may be a patient associated identifier, such as a label for a wristband, a label for inclusion in a medical record, etc.

In those embodiments where an actuator is configured to read non-active agent delivery component identifiers, the actuator may be configured to be modulated in a variety of different ways based on information received by the actuator from such identifiers. For example, a device may be configured such that an actuator reads identifiers from both an active agent delivery component and a patient, and actuates the delivery device only when information received from the identifiers matches, e.g., as determined by an algorithm, which may be present on the actuator or on a networked device. For example, in some instances a device is configured so that it can only be activated if the following occur: 1) the actuator scans an identifier, such as a barcode/RFID tag/NFC tag, associated with a patient (e.g., present on a wristband); the actuator scans an identifier, such as a barcode/RFID/NFC tag, affixed to the active agent delivery component; and 3) information received from the identifiers is determined to match, e.g., the patient is one that has been prescribed the active agent present in the active agent delivery component, such that the device is activated and is able to deliver the active agent to the patient. In those situations where the information from the identifiers is determined not to match, e.g., information obtained from the patient identifier states that an active agent different from that in the active agent delivery component is to be administered to a patient, the device is not activated and is unable to deliver the therapeutic agent to the patient.

In some instances, the actuator component actively communicates with an auto-generated barcode/RFID/NFC tag generated inside the patient's electronic medical record (EMR) after a drug specific order has been placed. Device activation in such embodiments includes communication with a physically associated patient identifier; a patient EMR associated identifier and the active agent delivery component identifier.

In some instances, activating the device through scanning a patient barcode and scanning/affixing the active agent delivery component results in one or more of: an order being placed in the EMR, auto-generation of a procedure note, generation of a bill, triggering of a follow-up appointment, etc.

In some instances, the active agent delivery component determination comprises usage information. Usage information is data about actual use of the active agent delivery component in the delivery of an active agent composition thereof to a subject. Such usage information includes, but is not limited to: information that an active agent composition was delivered from an active agent delivery component (e.g., during an administration event), where such information may include temporal information, such as date and time information, etc.; location or geographical information, such as where the usage took place; information about the disease condition for which the active agent delivery component was employed; information about the subject with which the active agent delivery component was employed, where such information may include one or more of: age, sex, disease condition, unique identity, co-morbid conditions and concomitant medications, medical history, and the like. In some instances, unique identifiers could be removed from this information. In some instances, the information could be configured to be transmitted in compliance with The Health Insurance Portability and Accountability Act of 1996 (HIPAA).

A determination that includes usage information, e.g., as described above, may be employed in a variety of different applications. Examples of such applications include billing, reimbursement, and inventory management processes. For example, a system may allow billing (i.e., charging the user) upon use. Alternatively, or additionally, a system may facilitate generation of a reimbursement request from a patient's insurance provider. Such determinations may also be employed in user based inventory management protocols. For example, a system may initiate an order to replace the used delivery component. In one embodiment, a system may allow a user to set up preferences regarding local stocking of therapeutic agent delivery components, and a usage determination may be employed to identify when local stocks have fallen below a predetermined number (such as preferences input by a user into a system, e.g., via a data input device, such as a graphical user interface), at which point a system may alert a user that it is time to order replacement stock and/or automatically order replacement stock. Such determinations may be employed in manufacturer based inventory management protocols. For example, usage data from all users or subsets thereof (e.g., geographical subsets, etc.) may be employed by a manufacturer to prepare manufacturing schedules, manage supply chain logistics, distribution schedules, etc. The system may use geographical information to determine a shipment location.

A determination that includes usage information, e.g., as described above, may also be employed in patient management processes. Patient management processes are applications involved in the therapeutic regimen or treatment protocol for a particular patient. Examples of such applications include where usage information is employed in conjunction with a patient's medical record, such as a patient's electronic medical record. For example, a determination that includes usage information, such as described above, may facilitate incorporation into a patient's medical record (e.g., where the incorporation may be automatic, such as where the medical record is an electronic medical record), to readily track actual administrations to a given patient, to identify dosage administration errors, produce procedure or operative note, etc. Such a patient management process could facilitate information or follow up for the patient, such as providing information to the patient or his/her caregiver, facilitating a follow up appointment, sending reminders, etc. In some embodiments, the device can interact with a patient's personal cellular devices, such as cellular phone, either via an application on the phone or through direct communication with the patient's electronic medical record accessed via the cellular phone or other cellular device.

Aspects of the present disclosure further include non-transitory computer readable storage mediums having instructions, e.g., in the form of software, for practicing the subject methods. Computer readable storage mediums may be employed on one or more computers for complete automation or partial automation of a system for practicing methods described herein. In certain embodiments, instructions in accordance with the method described herein can be coded onto a computer-readable medium in the form of “programming”, where the term “computer readable medium” as used herein refers to any non-transitory storage medium that participates in providing instructions and data to a computer for execution and processing. Examples of suitable non-transitory storage media include a floppy disk, hard disk, optical disk, magneto-optical disk, CD-ROM, CD-R, magnetic tape, non-volatile memory card, ROM, DVD-ROM, Blue-ray disk, solid state disk such as USB flash drive, and network attached storage (NAS), whether or not such devices are internal or external to the computer. A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Java (Sun Microsystems, Inc., Santa Clara, Calif.), Visual Basic (Microsoft Corp., Redmond, Wash.), and C++ (AT&T Corp., Bedminster, N.J.), as well as any many others.

In some embodiments, computer readable storage media of interest include a computer program stored thereon, where the computer program when loaded on the computer includes instructions for making one or more determinations, e.g., as described above. The computer readable storage medium may be employed on one or more computer systems having a display and operator input device. Operator input devices may, for example, be a keyboard, mouse, device buttons, smart phone, and the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

In some instances, the networked device further includes one or more databases, which may be accessed by the instructions, e.g., by a therapeutic agent determination delivery component determination module, such as described above. Such databases may include a variety of different types of information, including but not limited to: therapeutic agent delivery component information; actuator component information; patient information; and operator information. For example, a database may include therapeutic agent delivery component information, such as but not limited to a list of individual therapeutic agent delivery components and unique identifiers thereof (e.g., part numbers), where the list may include additional information, such as historical information, usage information, etc. A database may include actuator component information, such as but not limited to a list of individual actuator components and unique identifiers thereof (e.g., part numbers), where the list may include additional information, such as historical information, usage information, etc. A database may include patient information, e.g., in the form of one or more patient medical records, such as patient electronic medical records. A database may include operator information, such as a list of personnel authorized to use to the device, etc.

In some instances, the instructions (e.g., in the form of software) are configured to enable a user to perform one or more subsequent applications, where such applications include, but are not limited to: therapeutic agent delivery control applications, billing applications, reimbursement application, inventory management applications, patient management applications, e.g., as described above. For example, an embodiment of such instructions may be configured to allow a user to order parts, such as actuator components and/or therapeutic agent delivery components. In some instances, software according to the invention is configured to allow a user to set automatic ordering preferences. For example, a user could choose among billing systems, such as buying now (i.e., for buy-and-bill inventory management) or consignment model, where purchase does not occur until time of use. The user could also choose among payment systems, such as credit card payment, account billing, online payment, bank electronic funds transfer, and the like, and provide payment information, such as one or more credit card numbers, billing addresses, and the like. The user could also set inventory management preferences which would trigger re-ordering based on usage data or level of remaining inventory. The user could indicate ordering preferences, including a preference for a particular branded product or to allow substitution of a generic product or biosimilar. The user could indicate shipping preferences, including one or more ship-to addresses, dates, times, or days of the week for preferred delivery acceptance, delivery instructions, preferred delivery service (UPS, FedEx, etc.), and the like. Such software could be configured to handle one or multiple stock-keeping units (SKUs). In some embodiments, multiple different therapeutic agent delivery components that have been filled with different therapeutic agents could be included.

Aspects of the invention include docking stations that are configured to dock an actuator component (and in some instances an active agent delivery component), and systems that include a docking station and an actuator component. A docking station is a base unit or analogous device that is configured to engage with an actuator component, e.g., as described above. When engaged with an actuator component, the docking station may perform one or more functionalities, which functionalities may include, but are not limited to: maintaining a pain mitigation system in a desirable state (for example maintain a cooling system at a desired temperature); transferring data between the actuator component and an external device; sanitizing the distal end of an actuator component; recharging a power source of an actuator component; communicating with a computer, server, or database, and the like. The docking station may include a single actuator component dock, (i.e., a site or location configured to engagingly receive an actuator), or two or more actuator component docks, such that the number of actuator docks in a docking station may, in some instances, range from 1 to 6, such as 2 to 4. Docking stations of the invention may have any convenient configuration. Docking stations may be configured as table top devices, wall mounted devices, floor devices, etc., as desired.

In addition to actuator component docks, the docking station may further include one or more active agent delivery component docks, where active agent delivery component docks are sites or locations of the docking station that are configured to engagingly receive a therapeutic agent delivery component, e.g., as described above. When engaged with a therapeutic agent delivery component, the docking station may perform one or more functionalities, which functionalities may include, but are not limited to: maintaining a pain mitigation system in a desirable state (for example maintain a cooling system at a desired temperature); maintaining the active agent delivery component in a desirable state (for example maintaining the active agent delivery component at a desired temperature); transferring data between the active agent delivery component and an external device; sanitizing the distal end of an active agent delivery component; reading the identifier, and the like. The number of different active agent delivery component docks in a given docking station may vary, wherein in some instances the number ranges from 1 to 100, such as 1 to 50, including 1 to 30, 1 to 12, and 1 to 6.

In order to provide different desired functionalities, the docking station may include a number of a different subsystems or components. For example, a docking station may include a cooling system, e.g., that is configured to maintain the temperature of docked actuator and/or active agent delivery components in a desired range. Examples of suitable cooling systems include, but are not limited, those described above. The docking station may include a communications module, e.g., for mediating data transfer between docked actuator and/or active agent delivery components and a module of the docking stations and/or an external device. The docking station may include a power module, e.g., for recharging a power source of a docked actuator. The docking station may include detector, e.g., for detecting docked actuator and/or therapeutic agent delivery components. The docking station may include an identifier reader, e.g., for reading an identifier on a docked actuator and/or therapeutic agent delivery components, such as an identifier reader as described above. Where desired, the docking station may include an identifier or identifier reader, such as described above.

In addition to docking stations, e.g., as described above, aspects of the invention further include docking systems. Docking systems include a docking station having one or more actuator components and/or active agent delivery components docked therewith.

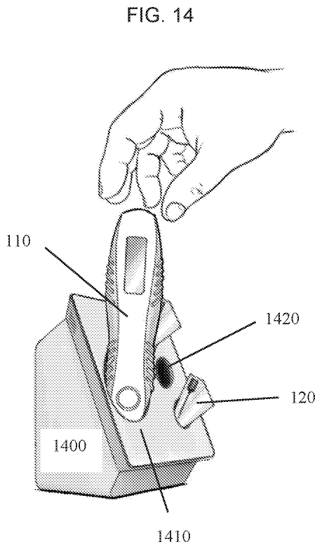

FIG. 14 provides a view of a table top docking station system that may be employed with the components of the delivery device shown in FIGS. 1A and 1B. As illustrated in FIG. 14, table top docking station 1400 includes an actuator component dock 1410 (shown dockingly engaged with an actuator component 110) and three therapeutic agent delivery docks 1420 (two of which are shown dockingly engaged with a therapeutic agent delivery component 120).

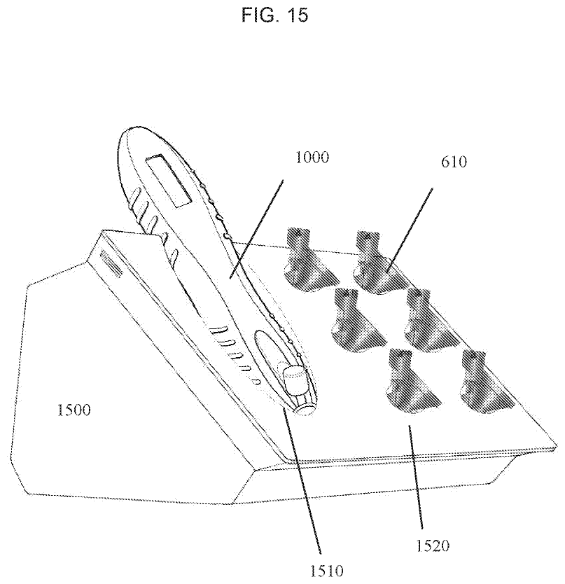

FIG. 15 provides a view of a docking station according to another embodiment of the invention. As shown in FIG. 15, docking station 1500 includes an actuator component dock 1510 (shown dockingly engaged with an actuator component 1000) and six therapeutic agent delivery docks 1520 (which are shown dockingly engaged with active agent delivery components 610).

### Methods

Aspects of the invention further include methods of delivering an active, e.g., therapeutic, agent to a target tissue delivery site of a subject using an active agent delivery devices of the invention. Aspects of the methods include: contacting a tissue contacting end of an active agent delivery device, e.g., as described above, to the target tissue delivery site; and actuating the therapeutic agent delivery system to deliver a therapeutic agent to the target tissue delivery site.

As reviewed above, the target tissue delivery site may vary. Examples of target tissue delivery sites include both external and internal delivery sites, wherein internal delivery sites include those sites located in body cavities. External sites include keratinized sites, as well as sites characterized by cutaneous membranes, mucous membranes, and tissue of the mucocutaneous zone. In some instances, the target tissue delivery site is an ocular site, where ocular sites of interest include a region that begins at the corneal limbus and extends anywhere from 1 mm to 10 mm posterior to the limbus, such as 2 mm to over 8 mm posterior to the limbus. In some instances, the area of interest includes the cornea and the corneal limbus.

To contact the distal end of the device with the target tissue site, the device may be manipulated so that the distal end of the device contacts the target tissue site. Where desired, contact of the distal end with the target tissue delivery site may be maintained by urging the distal end against the target tissue delivery site with moderate force. In some instances, the tissue-contacting portion of the device is sterile.

When present, the methods may further include actuating a pain mitigation system, e.g., prior to active agent delivery. The pain mitigation system is actuated to mitigate pain at the target tissue delivery site. Depending on the nature of the pain mitigation system, the pain mitigation system may be activated before or after contact of the distal end of the device with the target tissue delivery site. For example, where the pain mitigation system is a cooling system, the pain mitigation system may be activated so that a tissue engager of the cooling system is at a desired temperature prior to contact of the distal end of the device, and tissue engager, with the target tissue delivery site. Alternatively, where the pain mitigation system provides for pain mitigation via another mechanism, the pain mitigation system may be activated after contact of the distal end of the device with the target tissue delivery site.

Following tissue contact and, optionally, pain mitigation at the target tissue delivery site, the active agent delivery system is actuated to deliver an amount, e.g., dosage, of an active agent to the target tissue delivery site. Where actuation of the active agent delivery system results in automated therapeutic agent delivery, the device is held in such a manner such that contact of the distal end of the device with the target tissue delivery site is maintained during the active, e.g., therapeutic, agent delivery. Following active agent delivery, an injector of the active agent delivery device may be withdrawn from the target tissue delivery site, e.g., using a controlled retraction profile.

The devices may be employed to deliver an active, e.g., therapeutic, agent to a target tissue delivery site of different types of subjects. Generally such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In certain embodiments, the subjects are humans. The methods may be diagnostic and/or therapeutic methods.

In some instances, the methods include assembling a therapeutic agent delivery device, e.g., by release-ably engaging a therapeutic agent delivery component with an actuator component to produce a complete device, e.g., as described above. In some instances, the assembling includes contacting the distal end of the actuator component with a proximal end of the therapeutic delivery component that is present in a dock of a docking station, such as described above, where the contacting results in release-ably engaging the therapeutic agent delivery component with the actuator component such that the two components are operably coupled. In some instances, the release-ably engaging includes removing the actuator component from a docking station, such as described above. In some instances, the method further includes disengaging the therapeutic agent delivery component from distal end of the actuator component. The disengaging may include disposing the therapeutic agent delivery component. In some instances, the method further includes docking the actuator component in the docking station.

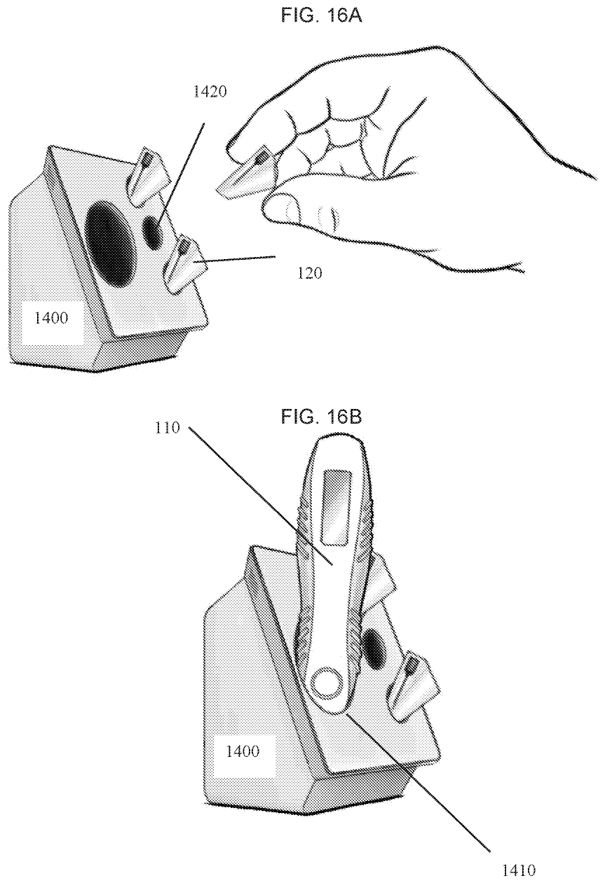

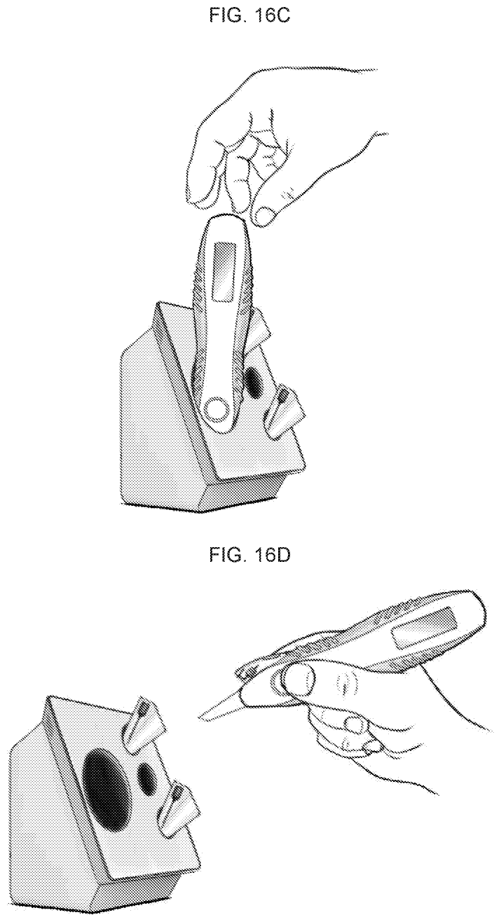

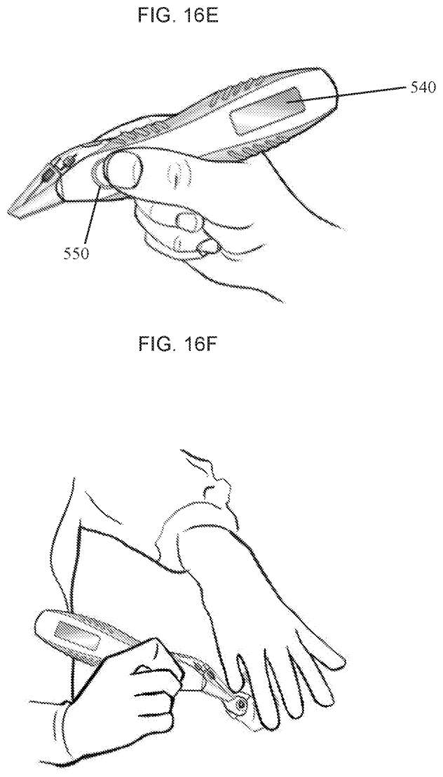

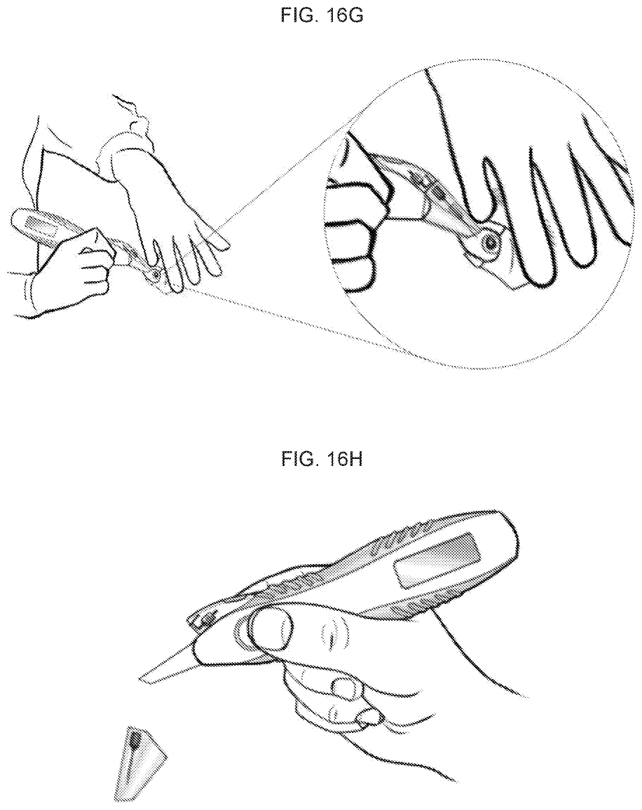

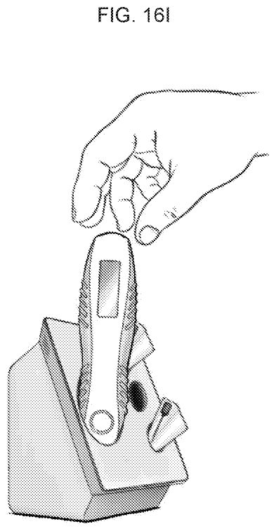

FIGS. 16A to 16I provide depictions of various steps of a therapeutic agent delivery workflow according an embodiment of the invention. In FIG. 16A, therapeutic agent delivery components 120 pre-loaded with a therapeutic agent composition are docked into therapeutic agent delivery component docks 1420 of a docking station 1400. In FIG. 16B, an actuator component is also placed in an actuator component dock of the docking station. As illustrated in FIG. 16C, docking station 1400 includes a cooling system, e.g., as described above, such as a liquid cooling system, that maintains one or both of the actuator and therapeutic agent delivery components at a desired temperature, e.g., a temperature ranging from 0 to −25, such as −10 to −15° C. In FIG. 16D, actuator 110 is removed from the docking station 1400. To assemble the device, the distal end of the actuator component 110 is inserted into the active agent delivery component 120 to produce the full assembled device 100 as shown in FIG. 16E. As shown in FIG. 16E, power button 550 may be depressed to activated device, and a message may be displayed on display 540 that the device is ready for use.

Following assembly of the ready to use device, e.g., as shown in FIG. 16E, the distal and of the assembled device is then positioned at the desired ocular tissue delivery site and the start button of the actuator is depressed to initial cooling of the ocular tissue delivery to achieve the desired anesthesia, as illustrated in FIG. 16F. While parameters may vary, in some instances the cooling tip of the device is maintained at a temperature ranging from 5 to −20, such as −5 to −10° C. for a period of time ranging from 5 to 120 s, such as 10 to 20 s to achieve the desired anesthesia at the ocular tissue delivery site. Following achievement of the desired anesthesia at the ocular tissue delivery site, the device may provide a single, such as an audio signal and or visual signal on the display, that the desired cooling has been provided.

Next, button 550 is depressed to commence therapeutic agent delivery to the now anesthetized ocular tissue delivery site, as shown in FIG. 16G. Upon depressing button 550, micro-motor actuator of the actuator component commences advancement of the needle into the ocular tissue delivery site, following which active, e.g., therapeutic, agent present in the reservoir is dispensed through the need. The needle is then retracted back into the therapeutic agent delivery component following delivery of the therapeutic agent.

Finally, as illustrated in FIGS. 16H and 16I, the therapeutic agent delivery component may be ejected from the actuator component, and the actuator component then placed back in the actuator component dock of the docking station.

### Utility

Devices of the invention, e.g., as described above, find use in the delivery of a variety of different types of therapeutic agents to a target tissue delivery site to treat a variety of different types of conditions. The therapeutic agent delivery devices of the invention may be used to deliver a therapeutic agent to a variety of target tissue delivery sites. Examples of target tissue delivery sites include both external and internal delivery sites, wherein internal delivery sites include those sites located in body cavities. External sites may include keratinized sites, as well as sites characterized by cutaneous membranes, mucous membranes, and tissue of the mucocutaneous zone. In some instances, the target tissue delivery site is an ocular tissue delivery site, where ocular tissue delivery sites of interest include a region that begins at the corneal limbus and extends anywhere from 2 mm to over 8 mm posterior to the limbus, such as 3 mm to 6 mm from the corneal limbus, e.g., 3 to 4 mm from the corneal limbus, e.g., to allow intraocular injection via pars plana or pars plicata. Ocular tissue delivery sites may include conjunctiva, episclera, and sclera of the eye. In some instances, the subject devices are used for intravitreal injection therapy (IVT), retrobulbar injection therapy, subtenon injection therapy, subretinal injection therapy, suprachoroial injection, subconjunctival injection therapy, intracameral injection therapy, and the like.

Examples of active, e.g., therapeutic, agents that may be delivered using devices of the invention include, but are not limited to steroids such as corticosteroids including dexamethasone (e.g., Ozurdex™), fluocinolone (e.g., Retisert™ or Iluvien™, loteprednol, difluprednate, fluorometholone, prednisolone, medrysone, triamcinolone, betamethasone and rimexolone; nonsteroidal anti-inflammatory agents such as salicylic-, indole acetic-, aryl acetic-, aryl propionic- and enolic acid derivatives including bromfenac, diclofenac, flurbiprofen, ketorolac tromethamine and nepafenac; antibiotics including azithromycin, bacitracin, besifloxacin, ciprofloxacin, erythromycin, gatifloxacin, gentamicin, levofloxacin, moxifloxacin, ofloxacin, sulfacetamide and tobramycin; VEGF inhibitors such as tyrosine kinase inhibitors, antibodies to VEGF, antibody fragments to VEGF, VEGF binding fusion proteins (e.g., pegaptinib, ranibizumab, bevacizumab, aflibercept, brolucizumab, Abicipar); PDGF inhibitors, antibodies to PDGF, antibody fragments to PDGF, PDGF binding fusion proteins (e.g., (Fovista™); anti-Factor D (lampalizumab), anti-TNF alpha agents such as antibodies to TNF-alpha, antibody fragments to TNF-alpha and TNF binding fusion proteins including infliximab, etanercept, adalimumab, certolizumab and golimumab; mTOR inhibitors such as sirolimus (Opsiria™), sirolimus analogues, Everolimus, Temsirolimus and mTOR kinase inhibitors; gases such as air, SF6, C2F6, C3F8, and others used in, for example, pneumatic retinopexy and retinal detachment repair; cells such as mesenchymal cells (e.g. mesenchymal stem cells), or cells transfected to produce a therapeutic compound; neuroprotective agents such as antioxidants, calcineurin inhibitors, NOS inhibitors, sigma-1 modulators, AMPA antagonists, calcium channel blockers and histone-deacetylases inhibitors; antihypertensive agents such as prostaglandin analogs, beta blockers, alpha agonists, and carbonic anhydrase inhibitors; aminosterols such as squalamine; antihistamines such as H 1-receptor antagonists and histamine H2-receptor antagonists; therapeutic cells; tyrosine kinase inhibitors and nucleic acid based therapeutics such as gene vectors, complement inhibitors; chemotherapeutic agents; insulin; plasmids and siRNA.

The device may be employed to deliver a therapeutic agent to treat a variety of different disease conditions. Disease conditions of interest include, but are not limited to, ocular conditions, such as ocular disease conditions, such as intraocular neovascular disease conditions. An “intraocular neovascular disease” is a disease characterized by ocular neovascularisation. Examples of intraocular neovascular diseases include, for example, proliferative retinopathies, choroidal neovascularization (CNV), age-related macular degeneration (AMD), geographic atrophy (GA), diabetic and other ischemia-related retinopathies, diabetic macular edema, pathological myopia, von Hippel-Lindau disease, histoplasmosis of the eye, Central Retinal Vein Occlusion (CRVO), Branch Retinal Vein Occlusion (BRVO), pterygium, corneal neovascularization, and retinal neovascularization. The term “age-related macular degeneration” refers to a medical condition which usually affects older adults and results in a loss of vision in the center of the visual field (the macula) because of damage to the retina. Some or all of these conditions can be treated by intravitreal injection of a VEGF-antagonist, e.g., as described above. Other ocular conditions that may be treated in accordance with aspects of the invention include, but are not limited to: retinal detachments (pneumatic retinopexy and retinal detachment repair via pars plana vitrectomy), by using devices of the invention to inject a gas into the eye, where the device may control the depth of injection to a desired/optimal depth. Disease conditions of interest also include central serous chorioretinopathy, uveitis, including anterior uveitis, pars planitis, intermediate uveitis, posterior uveitis, and glaucoma

### Kits

Also provided are kits that include at least one or more therapeutic agent delivery components, e.g., as described above. A kit may further include, where desired, an amount of a therapeutic agent, e.g., to be loaded in the therapeutic agent delivery component, a loading mechanism, e.g., a syringe, for loading the agent into the therapeutic agent delivery component (e.g., under sterile or aseptic conditions), an actuator component, a docking station, etc. The kit components may be present in packaging, which packaging may be sterile, as desired.

Also present in the kit may be instructions for using the kit components. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g., portable flash drive, DVD- or CD-ROM, etc. The instructions may take any form, including complete instructions for how to use the device or as a website address with which instructions posted on the world wide web may be accessed.

Notwithstanding the appended clauses, the disclosure set forth herein is also defined by the following clauses:

1. A therapeutic agent delivery device for delivering a therapeutic agent to a target tissue delivery site, the device comprising:

a therapeutic agent delivery component comprising a therapeutic agent delivery system; and

an actuator component comprising a body having a proximal end and a distal end, wherein the therapeutic agent delivery component is release-ably engaged to the distal end of the actuator component and the actuator component comprises:
a therapeutic agent delivery system actuator configured to actuate the therapeutic agent delivery system; and
a pain mitigation system configured to mitigate pain at the target tissue delivery site.
   2. The device according to Clause 1, wherein the pain mitigation system comprises an anesthesia producing system.
   3. The device according to Clause 2, wherein the anesthesia producing system comprises a cooling system.
   4. The device according to Clause 3, wherein the cooling system comprises a cooling system selected from the group consisting of: thermoelectric cooling system, liquid evaporation cooing system, Joule-Thompson cooling system, thermodynamic cycle cooling system, an endothermic reaction cooling system and a low-temperature substance cooling system.
   5. The device according to Clause 4, wherein the cooling system comprises a thermoelectric cooling system.
   6. The device according to Clause 1, wherein the pain mitigation system comprises an analgesia producing system.
   7. The device according to any of the preceding clauses, wherein the therapeutic agent delivery system comprises a therapeutic agent reservoir operably engaged with a tissue injector.
   8. The device according to Clause 7, wherein the tissue injector comprises a needle or cannula.
   9. The device according to any the preceding clauses, wherein the therapeutic agent is a liquid, semi-solid or solid.
   10. The device according to any of the preceding clauses, wherein the therapeutic agent delivery component comprises a tissue engager of the pain mitigation system.
   11. The device according to any of Clauses 1 to 9, wherein the therapeutic agent delivery component comprises a passage configured to accommodate a tissue engager of the pain mitigation system.
   12. The device according to any of Clauses 10 to 11, wherein the tissue engager comprises a thermally conductive member.
   13. The device according to any of the preceding clauses, wherein the device further comprises a locking element for release-ably engaging the therapeutic agent delivery component to the distal end of the actuator component of the device.
   14. The device according to any of the preceding clauses, wherein the therapeutic agent delivery component comprises an identifier and the actuator component comprises an identifier reader.
   15. The device according to Clause 14, wherein the device is configured to be active only when the identifier reader detects an acceptable identifier.
   16. The device according to any of the preceding clauses, wherein the device further comprises a communications module.
   17. The device according to Clause 16, wherein the communications module is configured for wireless communication.
   18. The device according to Clauses 16 or 17, wherein the communications module is configured for wired communication with the actuator component is docked in a docking station.
   19. The device according to any of the preceding clauses, wherein the device is a handheld device.
   20. The device according to any of the preceding clauses, wherein the therapeutic agent delivery component is sterile or aseptic.
   21. The device according to any of the preceding clauses, wherein the target tissue delivery site comprises an ocular tissue delivery site.
   22. A therapeutic agent delivery component configured to be release-ably engaged with a distal end of an actuator component of a therapeutic agent delivery device, the therapeutic agent delivery component comprising:

a body having a distal tissue contacting end and a proximal actuator engaging end; and

a therapeutic agent delivery system.

23. The therapeutic agent delivery component according to Clause 22, wherein the therapeutic agent delivery system comprises a therapeutic agent reservoir operably engaged with a tissue injector.
24. The therapeutic agent delivery component according to Clause 23, wherein the tissue injector comprises a needle or cannula.
25. The therapeutic agent delivery component according to any of Clauses 23 to 24, wherein tissue injector is configured to prevent contact with an ocular lens or ocular retina.
26. The therapeutic agent delivery component according to any of the preceding clauses, wherein the therapeutic agent delivery system comprises a therapeutic agent.
27. The therapeutic agent delivery component according to Clause 26, wherein the therapeutic agent is a liquid, semi-solid or solid.
28. The therapeutic agent delivery component according to any of Clauses 26 to 27, wherein therapeutic agent delivery system comprises a minimal volume of air.
29. The therapeutic agent delivery component according to Clause 28, wherein the minimal volume is 10 μl or less.
30. The therapeutic agent delivery component according to any of Clauses 26 to 29, wherein the amount of therapeutic agent present in the therapeutic agent delivery system is greater than the amount of therapeutic agent that is delivered from the therapeutic agent delivery component upon use.
31. The therapeutic agent delivery component according to any of Clauses 23 to 30, further comprising a port for introducing a therapeutic agent into the therapeutic agent reservoir.
32. The therapeutic agent delivery component according to any of Clauses 23 to 31, further comprising a window providing a view of the therapeutic agent reservoir.
33. The therapeutic agent delivery component according to any of Clauses 22 to 32, wherein the therapeutic agent delivery system comprises a filter.
34. The therapeutic agent delivery component according to any of Clauses 22 to 33, wherein the therapeutic agent delivery component comprises a tissue engager of a pain mitigation system.
35. The therapeutic agent delivery component according to any of Clauses 22 to 33, wherein the therapeutic agent delivery component comprises a passage configured to accommodate a tissue-engager of a pain mitigation system.
36. The therapeutic agent delivery component according to Clause 35, wherein the therapeutic agent delivery component comprises a membrane at the distal end of the passage that is displaced upon occupation of the passage by the tissue-engager.
37. The therapeutic agent delivery component according to any of Clauses 34 to 36, wherein the tissue engager comprises a thermally conductive member.
38. The therapeutic agent delivery component according to any of Clauses 22 to 37, further comprising a locking element for release-ably engaging the proximal actuator engaging end to the distal end of the actuator component.
39. The therapeutic agent delivery component according to any of Clauses 22 to 38, further comprising a removable cover at the tissue contacting end.
40. The therapeutic agent delivery component according to Clause 39, wherein the removable cover is sterile or sanitized.
41. The therapeutic agent delivery component according to any of Clauses 22 to 40, further comprising an opening at the tissue contacting end configured to provide access of the therapeutic agent delivery system to a target tissue.
42. The therapeutic agent delivery component according to any of Clauses 22 to 41, further comprising an identifier.
43. The therapeutic agent delivery component according to Clause 42, wherein the identifier is configured to be read by an identifier reader of the actuator component.
44. The therapeutic agent delivery component according to Clause 42, wherein the identifier is visual identifier configured to be read by a health care practitioner.
45. The therapeutic agent delivery component according to Clause 44, wherein the visual identifier conveys information about the therapeutic agent delivery component or therapeutic agent present therein.
46. The therapeutic agent delivery component according to any of Clauses 22 to 45, further comprising an antimicrobial element.
47. The therapeutic agent delivery component according to Clause 46, wherein the antimicrobial element comprises an antimicrobial agent.
48. The therapeutic agent delivery component according to Clause 47, wherein the antimicrobial agent is present in a matrix.
49. The therapeutic agent delivery component according to Clause 47, wherein the antimicrobial agent is present in a reservoir.
50. The therapeutic agent delivery component according to Clause 26, wherein the antimicrobial agent is present as a semi-solid composition.
51. The therapeutic agent delivery component according to any of Clauses 22 to 50, further comprising an analgesic/anesthetic agent.
52. The therapeutic agent delivery component according to Clause 51, wherein the analgesic/anesthetic agent is present in a matrix.
53. The therapeutic agent delivery component according to Clause 51, wherein the analgesic/anesthetic agent is present in a reservoir.
54. The therapeutic agent delivery component according to Clause 51, wherein the analgesic/anesthetic agent is present as a semi-solid composition.
55. The therapeutic agent delivery component according to any of Clauses 22 to 54, wherein at least a portion of the therapeutic agent delivery component is reusable.
56. The therapeutic agent delivery component according to any of Clauses 22 to 55, wherein the tissue contacting end is configured to contact ocular tissue.
57. An actuator component of a therapeutic agent delivery device, the actuator component comprising:

a body having a proximal end and a distal end, wherein the distal end is configured to be release-ably engaged to a therapeutic agent delivery component; a therapeutic agent delivery system actuator configured to actuate a therapeutic agent delivery system of the therapeutic agent delivery component; and

a pain mitigation system configured to mitigate pain in target tissue delivery site.

58. The actuator component according to Clause 57, wherein the therapeutic agent delivery system comprises a therapeutic agent reservoir operably engaged with a tissue injector.
59. The actuator component according to Clause 58, wherein the therapeutic agent delivery system actuator is configured to provide for control of one or more of angular position, linear position, velocity and acceleration of the tissue injector.
60. The actuator component according to any of Clauses 58 to 59, wherein the therapeutic agent delivery system actuator is configured to provide for control of release of a therapeutic agent from the therapeutic agent reservoir.
61. The actuator component according to any of Clauses 58 to 60, wherein the therapeutic agent delivery system actuator is configured to prime the tissue injector.
62. The actuator component according to any of Clauses 58 to 61, wherein the therapeutic agent delivery system actuator is configured to provide for controlled removal of the tissue injector from the target tissue delivery site.
63. The actuator component according to any of Clauses 58 to 62, wherein the therapeutic agent delivery system actuator comprises a motor.
64. The actuator component according to Clause 63, wherein the motor comprises a micro-motor.
65. The actuator component according to any of Clauses 57 to 63 wherein the therapeutic agent delivery system actuator comprises a non-motorized actuator.
66. The actuator component according to any of Clauses 57 to 65, wherein the pain mitigation system comprises an anesthesia producing system.
67. The actuator component according to Clause 66, wherein the anesthesia producing system comprises a cooling system.
68. The actuator component according to Clause 67, wherein the cooling system comprises a cooling system selected from the group consisting of: thermoelectric cooling system, liquid evaporation cooing system, Joule-Thompson cooling system, thermodynamic cycle cooling system, an endothermic reaction cooling system and a low-temperature substance cooling system.
69. The actuator component according to Clause 68, wherein the cooling system comprises a thermoelectric cooling system.
70. The actuator component according to any of Clauses 57 to 65, wherein the pain mitigation system comprises an analgesia producing system.
71. The actuator component according to any of Clauses 57 to 70, wherein the pain mitigation system comprises tissue engager.
72. The actuator component according to Clause 71, wherein the tissue engager comprises a thermally conductive member.
73. The actuator component according to any of Clauses 57 to 72, further comprising a locking element for release-ably engaging a therapeutic agent delivery component to the distal end of the actuator component.
74. The actuator component according to any of Clauses 57 to 73, further comprising an identifier reader for reading an identifier of a therapeutic agent delivery component release-ably engaged with the actuator component.
75. The actuator component according to Clause 74, wherein the actuator component is configured to be active only when the identifier reader detects an acceptable identifier.
76. The actuator component according to any of Clauses 57 to 75, further comprising a communications module.
77. The according component according to Clause 76, wherein the communications module is configured for wireless communication.
78. The actuator component according to Clauses 76 to 77, wherein the communications module is configured for wired communication when the actuator component is docked in a docking station.
79. The actuator component according to any of Clauses 57 to 78, wherein the actuator component is configured to be handheld.
80. The actuator component according to any of Clauses 57 to 79, wherein the actuator comprises a display.
81. The actuator component according to any of Clauses 57 to 80, wherein the therapeutic agent delivery device is configured to deliver a therapeutic agent to an ocular tissue delivery site.
82. A system comprising:

a docking station comprising:
an actuator dock configured to receive an actuator component according to any of Clauses 57 to 81; and
an actuator component according to any of Clauses 57 to 81 received in the actuator dock.
   83. The system according to Clause 82, wherein the docking station further includes:

a therapeutic agent delivery component dock configured to receive a therapeutic agent delivery component according to any of Clauses 22 to 56; and

a therapeutic agent delivery component according to any of Clauses 22 to 56 received in the therapeutic agent delivery component dock.

84. The system according to Clause 83, wherein the system comprises a plurality of therapeutic agent delivery component docks.
85. The system according to Clause 84, wherein the system comprises 5 to 15 therapeutic agent delivery component docks.
86. The system according to any of Clauses 82 to 85, wherein the docking station comprises a cooling system configured to maintain at least one of an actuator component and a therapeutic agent delivery component at a predetermined temperature.
87. The system according to any Clauses 82 to 86, wherein the docking station comprises a communications module.
88. The system according to Clause 87, wherein the communications module is configured for wireless communication.
89. The system according to Clause 87, wherein the communications module is configured for wired communication.
90. The system according to any of Clauses 82 to 89, wherein the docking station comprises a detector for detecting an identifier on one or more of:

an actuator component docked with the docking station; and

a therapeutic agent delivery component docked with the docking station.

91. The system according to any of Clauses 82 to 90, wherein the system further comprises a visual identifier.
92. A docking station comprising:

an actuator dock configured to receive an actuator component according to any of Clauses 57 to 81.

93. The docking station according to Clause 92, wherein the docking station further comprises a therapeutic agent delivery component dock configured to receive a therapeutic agent delivery component according to any of Clauses 22 to 56.
94. The docking station according to Clause 93, wherein the docking station comprises a plurality of therapeutic agent delivery component docks.
95. The docking station according to Clause 94, wherein the docking station comprises 5 to 15 therapeutic agent delivery component docks.
96. The docking station according to any Clauses 92 to 95, wherein the docking station comprises a cooling system configured to maintain at least one of an actuator component and a therapeutic agent delivery component a predetermined temperature.
97. The docking station according to any of Clauses 92 to 96, wherein the docking station comprises a communications module.
98. The docking station according to Clause 97, wherein the communications module is configured for wired communication.
99. The docking station according to Clause 97, wherein the communications module is configured for wireless communication.
100. The docking station according to any of Clauses 92 to 99, wherein the docking station comprises a detector for detecting an identifier on one or more of an actuator component and a therapeutic agent delivery component.
101. A method of delivering a therapeutic agent to a target tissue delivery site, the method comprising:

(a) contacting a tissue contacting end of therapeutic agent delivery device to the target tissue delivery site, wherein the therapeutic agent delivery device comprises:
a therapeutic agent delivery component comprising the tissue contacting end and a therapeutic agent delivery system; and
an actuator component comprising a body having a proximal end and a distal end, wherein the therapeutic agent delivery component is release-ably engaged to the distal end of the actuator component and the actuator component comprises:
   a therapeutic agent delivery system actuator configured to actuate the therapeutic agent delivery system; and
   a pain mitigation system configured to mitigate pain at the target tissue delivery site;

(b) actuating the pain mitigation system to mitigate pain at the target tissue delivery site; and

(c) actuating the therapeutic agent delivery system to deliver a therapeutic agent to the target tissue delivery site.

102. The method according to Clause 101, wherein the pain mitigation system comprises an anesthesia producing system and the method comprises producing anesthesia at the target tissue delivery site.
103. The method according to Clause 102, wherein the anesthesia producing system comprises a cooling system.
104. The method according to Clause 103, wherein the cooling system comprises a cooling system selected from the group consisting of: thermoelectric cooling system, liquid evaporation cooling system, Joule-Thompson cooling system, thermodynamic cycle cooling system, an endothermic reaction cooling system and a low-temperature substance cooling system.
105. The method according to Clause 104, wherein the cooling system comprises a thermoelectric cooling system.
106. The method according to Clause 101, wherein the pain mitigation system comprises an analgesia producing system and the method comprises producing analgesia at the target tissue delivery site.
107. The method according to any of Clauses 100 to 106, wherein the therapeutic agent delivery system comprises a therapeutic agent reservoir operably engaged with a tissue injector.
108. The method according to Clause 107, wherein the tissue injector comprises a needle or cannula.
109. The therapeutic agent delivery component according to any of Clauses 107 to 108, wherein tissue injector is configured to prevent contact with an ocular lens or ocular retina.
110. The method according to any of Clauses 100 to 109, wherein the therapeutic agent delivery component comprises a tissue engager of the pain mitigation system.
111. The method according to any of Clauses 100 to 109, wherein the therapeutic agent delivery component comprises a passage configured to accommodate a tissue engager of the pain mitigation system.
112. The method according to any of Clauses 110 to 111, wherein the tissue engager comprises a thermally conductive member.
113. The method according to any of Clauses 100 to 112, wherein the device further comprises a locking element for release-ably engaging the therapeutic agent delivery component to the distal end of the actuator component of the device.
114. The method according to any of Clauses 100 to 113, wherein the therapeutic agent delivery component comprises an identifier and the actuator component comprises an identifier reader.
115. The method according to Clause 114, wherein the device is configured to be active only when the identifier reader detects an acceptable identifier.
116. The method according to any of Clauses 100 to 115, wherein the device further comprises a communications module.
117. The method according to Clause 116, wherein the communications module is configured for wireless communication.
118. The method according to any of Clauses 116 to 117, wherein the method further comprises transmitting data between the device and a receiver.
119. The method according to any of Clauses 100 to 118, wherein the device is a handheld device.
120. The method according to any of Clauses 100 to 119, wherein the therapeutic agent delivery component is sterile or aseptic.
121. The method according to any of Clauses 100 to 120, wherein the method further comprises release-ably engaging the therapeutic agent delivery component to the distal end of the actuator component.
122. The method according to Clause 121, wherein the release-ably engaging comprises removing the actuator component from a docking station.
123. The method according to Clause 121, wherein the release-ably engaging further comprises coupling the removed actuator to a therapeutic agent delivery component that is docked in the docking station.
124. The method according to any of Clauses 100 to 123, wherein the method further comprises disengaging the therapeutic agent delivery component from distal end of the actuator component.
125. The method according to Clause 124, wherein the method further comprises docking the actuator component in the docking station.
126. The method according to any of Clauses 100 to 125, wherein the target tissue delivery site comprises an ocular tissue delivery site.
127. The method according to Clause 126, wherein the method is a method of treating a subject for an ocular disease.
128. A kit comprising:

a therapeutic agent delivery component according to any of Clauses 22 to 56; and

sterile packaging.

129. The kit according to Clause 128, wherein the kit comprises a plurality of therapeutic agent delivery components according to any of Clauses 22 to 56.
130. The kit according to any of Clauses 128 to 129, wherein the kit further comprises a therapeutic agent composition.

Notwithstanding the appended clauses, the disclosure is also defined by the following additional clauses:

1. A therapeutic agent delivery device for delivering an active agent to a target tissue delivery site, the device comprising:

(a) a therapeutic agent delivery component comprising:
(i) a therapeutic agent delivery system; and
(ii) an identifier; and

(b) an actuator component comprising:
(i) a body having a proximal end and a distal end;
(ii) a therapeutic agent delivery system actuator configured to actuate the therapeutic agent delivery system; and
(iii) an identifier reader;

wherein the therapeutic agent delivery component is release-ably engaged to the distal end of the actuator component.

2. The device according to Clause 1, wherein the identifier comprises a barcode.
3. The device according to Clause 2, wherein the barcode comprises a linear barcode.
4. The device according to Clause 2, wherein the barcode comprises a matrix barcode.
5. The device according to Clause 1, wherein the identifier comprises a radio frequency identification (RFID) tag.
6. The device according to Clause 5, wherein the RFID tag comprises a near-field communication (NFC) tag.
7. The device according to any of the preceding clauses, wherein the device further comprises a communications module.
8. The device according to Clause 7, wherein the communications module is configured for wired communication.
9. The device according to Clause 7, wherein the communications module is configured for wireless communication.
10. The device according to any of the preceding clauses, wherein the therapeutic agent delivery device is configured to be active only when the identifier reader detects an acceptable identifier.
11. The device according to any of the preceding clauses, wherein the identifier comprises therapeutic agent delivery component information.
12. The device according to Clause 11, wherein the therapeutic agent delivery component information comprises therapeutic agent delivery component historical information.
13. The device according to Clause 11, wherein the therapeutic agent delivery component historical information comprises one or more of: a therapeutic agent identifier; therapeutic agent dose, concentration and/or volume information; a manufacturing lot number for the therapeutic agent delivery component or a therapeutic agent composition thereof; therapeutic agent delivery component handling information; and therapeutic agent delivery component expiration date.
14. The device according to any of the preceding clauses, wherein the therapeutic agent delivery device is configured to transmit information obtained by the identifier reader to a networked device.
15. The device according to Clause 14, wherein the information comprises use information.
16. The device according to Clause 15, wherein the use information comprises use date information.
17. The device according to any of Clauses 15 to 16, wherein the therapeutic agent delivery component use information comprises administration information.
18. The device according to any of the preceding clauses, wherein the therapeutic agent delivery system comprises a therapeutic agent reservoir in fluidic communication with a tissue injector.
19. The device according to Clause 18, wherein the tissue injector comprises a needle or cannula.
20. The device according to any of the preceding clauses, wherein the device further comprises a locking element for release-ably engaging the therapeutic agent delivery component to the distal end of the actuator component of the device.
21. The device according to any of the preceding clauses, wherein the device is a handheld device.
22. The device according to any of the preceding clauses, wherein the therapeutic agent delivery component is sterile.
23. The device according to any of the preceding clauses, wherein the device is configured to deliver a therapeutic agent to an ocular tissue delivery site.
24. A therapeutic agent delivery component configured to be release-ably engaged with a distal end of an actuator component of a therapeutic agent delivery device, the therapeutic agent delivery component comprising:

a body having a distal tissue contacting end and a proximal actuator engaging end;

a therapeutic agent delivery system; and

an identifier.

25. The therapeutic agent delivery component according to Clause 24, wherein the identifier comprises a barcode.
26. The therapeutic agent delivery component according to Clause 25, wherein the barcode comprises a linear barcode.
27. The therapeutic agent delivery component to Clause 25, wherein the barcode comprises a matrix barcode.
28. The therapeutic agent delivery component according to Clause 24, wherein the identifier comprises a radio frequency identification (RFID) tag.
29. The therapeutic agent delivery component according to Clause 28, wherein the RFID tag comprises a near-field communication (NFC) tag.
30. The therapeutic agent delivery component according to any of Clauses 24 to 29, wherein the identifier comprises therapeutic agent delivery component information.
31. The therapeutic agent delivery component according to Clause 30, wherein the therapeutic agent delivery component information comprises therapeutic agent delivery component historical information.
32. The therapeutic agent delivery component according to Clause 31, wherein the therapeutic agent delivery component historical information comprises one or more of: a therapeutic agent identifier; therapeutic agent dose, concentration and/or volume information; a manufacturing lot number for the therapeutic agent delivery component or a therapeutic agent composition thereof; therapeutic agent delivery component handling information; and therapeutic agent delivery component expiration date.
33. The therapeutic agent delivery component according to any of Clauses 24 to 32, wherein the therapeutic agent delivery system comprises a therapeutic agent reservoir in fluidic communication with a tissue injector.
34. The therapeutic agent delivery component according to Clause 33, wherein the tissue injector comprises a needle or cannula.
35. The therapeutic agent delivery component according to any of Clauses 24 to 34, wherein the therapeutic agent delivery component further comprises a locking element for release-ably engaging the therapeutic agent delivery component to the distal end of the actuator component.
36. The therapeutic agent delivery component according to any of Clauses 24 to 35, wherein the therapeutic agent delivery component is sterile.
37. The therapeutic agent delivery component according to any of Clauses 24 to 36, further comprising a sensor.
38. The therapeutic agent delivery component according to Clause 37, wherein the sensor is an environmental or locational sensor.
39. The therapeutic agent delivery component according to any of Clauses 24 to 38, wherein the therapeutic agent delivery system is configured to deliver a therapeutic agent to an ocular tissue delivery site.
40. An actuator component of a therapeutic agent delivery device, the actuator component comprising:

a body having a proximal end and a distal end, wherein the distal end is configured to be release-ably engaged to a therapeutic agent delivery component; a therapeutic agent delivery system actuator configured to actuate a therapeutic agent delivery system of the therapeutic agent delivery component; and

an identifier reader configured to read an identifier of a therapeutic agent delivery component release-ably engaged to the actuator component.

41. The actuator component according to Clause 40, wherein the identifier reader comprises a barcode scanner.
42. The actuator component according to Clause 40, wherein the identifier reader comprises a radio frequency identification (RFID) tag reader.
43. The actuator component according to any of Clauses 40 to 42, further comprising a communications module.
44. The actuator component according to Clause 43, wherein the communications module is configured for wireless communication.
45. The actuator component according to any of Clauses 40 to 44, wherein the actuator component is configured to be active only when the identifier reader detects an acceptable identifier.
46. The actuator component according to any of Clauses 40 to 45, wherein the actuator further comprises a locking element for release-ably engaging a therapeutic agent delivery component to the distal end of the actuator component.
47. The actuator component according to any of Clauses 40 to 45, wherein the actuator component is a handheld device.
48. The actuator component according to any of Clauses 40 to 46, wherein the actuator component is configured to read a non-therapeutic agent delivery component identifier.
49. The actuator component according to Clause 48, wherein the non-therapeutic agent delivery component identifier is a patient associated identifier.
50. The actuator component according to Clause 49, wherein the patient associated identifier is physically associated with a patient.
51. The actuator component according to Clause 49, wherein the patient associated identifier is provided by a patient medical record.
52. The actuator component according to Clause 51, wherein the patient medical record is an electronic medical record.
53. A system comprising:

(A) a therapeutic agent delivery device for delivering an active agent to a target tissue delivery site, the device comprising:
(1) a therapeutic agent delivery component comprising:
   (a) a therapeutic agent delivery system; and
   (b) an identifier; and
(2) an actuator component comprising:
   (a) a body having a proximal end and a distal end;
   (b) a therapeutic agent delivery system actuator configured to actuate the therapeutic agent delivery system;
   (c) an identifier reader; and
   (d) a first communication module;
wherein the therapeutic agent delivery component is release-ably engaged to the distal end of the actuator component; and
(B) a networked device comprising a second communication module communicably linked to the first communication module.
   54. The system according to Clause 53, wherein the identifier comprises a barcode.
   55. The system according to Clause 54, wherein the barcode comprises a linear barcode.
   56. The system according to Clause 54, wherein the barcode comprises a matrix barcode.
   57. The system according to Clause 53, wherein the identifier comprises a radio frequency identification (RFID) tag.
   58. The system according to Clause 57, wherein the RFID tag comprises a near-field communication (NFC) tag.
   59. The system according to any of Clauses 53 to 58, wherein the device is configured to be active only when the identifier reader detects an acceptable identifier.
   60. The system according to any of Clauses 53 to 59, wherein the identifier comprises therapeutic agent delivery component information.
   61. The system according to Clause 60, wherein the therapeutic agent delivery component information comprises therapeutic agent delivery component historical information.
   62. The system according to Clause 61, wherein the therapeutic agent delivery component historical information comprises one or more of: a therapeutic agent identifier; therapeutic agent dose, concentration and/or volume information; a manufacturing lot number for the therapeutic agent delivery component or a therapeutic agent composition thereof; therapeutic agent delivery component handling information; and therapeutic agent delivery component expiration date.
   63. The system according to any of Clauses 60 to 62, wherein the therapeutic agent delivery device is configured to transmit the therapeutic agent delivery component information to the networked device.
   64. The system according to any of Clauses 59 to 63, wherein the therapeutic agent delivery device is configured to transmit therapeutic agent delivery component use information to the networked device.
   65. The system according to Clause 64, wherein the use information includes use date information.
   66. The system according to Clause 64, wherein the use information includes administration information.
   67. The system according to any of Clauses 53 to 66, wherein the therapeutic agent delivery system comprises a therapeutic agent reservoir in fluidic communication with a tissue injector.
   68. The system according to Clause 67, wherein the tissue injector comprises a needle or cannula.
   69. The system according to any of Clauses 53 to 68, wherein the therapeutic agent delivery device further comprises a locking element for release-ably engaging the therapeutic agent delivery component to the distal end of the actuator component of the device.
   70. The system according to any of Clauses 53 to 69, wherein the therapeutic agent delivery device is a handheld device.
   71. The system according to any of Clauses 53 to 70, wherein the therapeutic agent delivery component is sterile.
   72. The system according to any of Clauses 53 to 71, wherein the device is configured to deliver a therapeutic agent to an ocular tissue delivery site.
   73. The system according to any of Clauses 53 to 72, wherein the networked device comprises an electronic medical record.
   74. The system according to any of Clauses 53 to 73, wherein the networked device comprises a therapeutic agent delivery component determination module.
   75. The system according to Clause 74, wherein the therapeutic agent delivery component determination module comprises:

a processor coupled to the second communication module; and

a memory coupled to the processor, the memory storing non-transitory computer-readable instructions that, when executed by the processor, cause the processor to make a therapeutic agent delivery component determination based on data received from the therapeutic agent delivery device.

76. The system according to Clause 75, wherein the therapeutic agent delivery component determination comprises therapeutic agent delivery component historical information.
77. The system according to Clause 76, wherein the therapeutic agent delivery component determination is configured to be employed in a therapeutic agent delivery device control process.
78. The system according to Clause 77, wherein the therapeutic agent delivery device control process controls actuation of the delivery device based on the therapeutic agent delivery component determination.
79. The system according to Clause 75, wherein the therapeutic agent delivery component determination comprises usage information.
80. The system according to Clause 79, wherein the therapeutic agent delivery component determination is configured to be employed in an inventory management process.
81. The system according to Clause 79, wherein the therapeutic agent delivery component determination is configured to be employed in a billing process.
82. The system according to Clause 79, wherein the therapeutic agent delivery component determination is configured to be employed in a reimbursement process.
83. The system according to Clause 79, wherein the therapeutic agent delivery component determination is configured to be employed in a patient management process.
84. The system according to any of Clauses 74 to 83, wherein the networked device comprises a database that is accessed by the therapeutic agent delivery component determination module.
85. The system according to Clause 84, wherein the database comprises one or more of: therapeutic agent delivery component information; actuator component information; patient information; and operator information.
86. A system comprising:

(a) a therapeutic agent delivery component comprising:
(i) a therapeutic agent delivery system; and
(ii) an identifier; and

(b) a networked device comprising a therapeutic agent delivery component determination module.

87. The system according to Clause 86, wherein the therapeutic agent delivery component determination module comprises:
(i) a processor configured to receive data from the identifier; and
(ii) a memory coupled to the processor, the memory storing non-transitory computer-readable instructions that, when executed by the processor, cause the processor to make a therapeutic agent delivery component determination based on data received from the identifier.
   88. The system according to any of Clauses 86 to 87, wherein the identifier comprises a barcode.
   89. The system according to Clause 88, wherein the barcode comprises a linear barcode.
   90. The system according to Clause 88, wherein the barcode comprises a matrix barcode.
   91. The system according to Clauses 86 to 87, wherein the identifier comprises a radio frequency identification (RFID) tag.
   92. The system according to Clause 91, wherein the RFID tag comprises a near-field communication (NFC) tag.
   93. The system according to any of Clauses 86 to 92, wherein the identifier comprises therapeutic agent delivery component information.
   94. The system according to Clause 93, wherein the therapeutic agent delivery component information comprises therapeutic agent delivery component historical information.
   95. The system according to Clause 94, wherein the therapeutic agent delivery component historical information comprises one or more of: a therapeutic agent identifier; therapeutic agent dose, concentration and/or volume information; a manufacturing lot number for the therapeutic agent delivery component or a therapeutic agent composition thereof; therapeutic agent delivery component handling information; and therapeutic agent delivery component expiration date.
   96. The system according to any of Clauses 93 to 95, wherein the therapeutic agent delivery component is configured to transmit the therapeutic agent delivery component information to the networked device.
   97. The system according to any of Clauses 86 to 96, wherein the therapeutic agent delivery system comprises a therapeutic agent reservoir in fluidic communication with a tissue injector.
   98. The system according to any of Clauses 86 to 97, wherein the therapeutic agent delivery component is configured for use in delivery of a therapeutic agent to an ocular tissue delivery site.
   99. The system according to any of Clauses 86 to 98, wherein the therapeutic agent delivery component determination comprises therapeutic agent delivery component historical information.
   100. The system according to Clause 99, wherein the therapeutic agent delivery component determination is configured to be employed in a therapeutic agent delivery device control process, and optionally wherein the therapeutic agent delivery device control process controls actuation of a delivery device based on the therapeutic agent delivery component determination.
   101. The system according to any of Clauses 86 to 100, wherein the therapeutic agent delivery component determination comprises usage information.
   102. The system according to Clause 101, wherein the therapeutic agent delivery component determination is configured to be employed in an inventory management process.
   103. The system according to Clause 101, wherein the therapeutic agent delivery component determination is configured to be employed in a billing process.
   104. The system according to Clause 101, wherein the therapeutic agent delivery component determination is configured to be employed in a reimbursement process.
   105. The system according to Clause 101, wherein the therapeutic agent delivery component determination is configured to be employed in a patient management process.
   106. The system according to any of Clauses 86 to 105, wherein the networked device comprises a database that is accessed by the therapeutic agent delivery component determination module.
   107. The system according to Clause 106, wherein the database comprises one or more of: therapeutic agent delivery component information; actuator component information; patient information; and operator information.
   108. A method of delivering a therapeutic agent to a target tissue delivery site, the method comprising:

(a) contacting a tissue-contacting end of therapeutic agent delivery device to the target tissue delivery site, wherein the therapeutic agent delivery device comprises:
a therapeutic agent delivery component comprising:
   a therapeutic agent delivery system; and
   an identifier; and
an actuator component comprising:
   a body having a proximal end and a distal end;
   a therapeutic agent delivery system actuator configured to actuate the therapeutic agent delivery system;
   and
   an identifier reader;
wherein the therapeutic agent delivery component is release-ably engaged to the distal end of the actuator component

(b) actuating the therapeutic agent delivery system to deliver a therapeutic agent to the target tissue delivery site.

109. The method according to Clause 108, wherein the device is configured to be active only when the identifier reader detects an acceptable identifier.
110. The method according to any of Clauses 108 and 109, wherein the actuator component further comprises a communications module and the method comprises transmitting data from the communications module to a networked device.
111. The method according to Clause 110, wherein data is wirelessly transmitted.
112. The method according to any of Clauses 110 and 111, wherein the data comprises therapeutic agent delivery component historical information.
113. The method according to any of Clauses 110 and 111, wherein the data comprises therapeutic agent delivery component use information.
114. The method according to any of Clauses 110 to 113, wherein the networked device comprises a therapeutic agent delivery component determination module.
115. The method according to Clause 114, wherein the therapeutic agent delivery component determination module comprises:
a processor coupled to the second communication module; and
a memory coupled to the processor, the memory storing non-transitory computer-readable instructions that, when executed by the processor, cause the processor to make a therapeutic agent delivery component determination based on data received from the therapeutic agent delivery device; and

the method further comprises receiving the therapeutic agent delivery component determination.

116. The method according to Clause 115, wherein the therapeutic agent delivery component determination comprises therapeutic agent delivery component historical information.
117. The method according to Clause 116, wherein the therapeutic agent delivery component historical information comprises one or more of: a therapeutic agent identifier; therapeutic agent dose, concentration and/or volume information; a manufacturing lot number for the therapeutic agent delivery component or a therapeutic agent composition thereof; therapeutic agent delivery component handling information; and therapeutic agent delivery component expiration date.
118. The method according to any of Clauses 115 to 117, wherein the method further comprises controlling actuation of the therapeutic agent delivery device based on the therapeutic agent delivery component determination.
119. The method according to Clause 115, wherein the therapeutic agent delivery component determination comprises usage information.
120. The method according to Clause 119, wherein the method further comprises employing the therapeutic agent delivery component determination in an inventory management process.
121. The method according to Clause 119, wherein the method further comprises employing the therapeutic agent delivery component determination in a billing process.
122. The method according to Clause 119, wherein the method further comprises employing the therapeutic agent delivery component determination in a reimbursement process.
123. The method according to Clause 119, wherein the method further comprises employing the therapeutic agent delivery component determination in a patient management process.
124. The method according to any of Clauses 108 to 123, wherein the method further comprises release-ably engaging the therapeutic agent delivery component to the distal end of the actuator component.
125. The method according to any of Clauses 108 to 124, wherein the method further comprises disengaging the therapeutic agent delivery component from distal end of the actuator component.
126. The method according to any of Clauses 108 to 125, wherein the target tissue delivery site comprises an ocular tissue delivery site.
127. The method according to Clause 126, wherein the method is a method of treating a subject for an ocular disease.
128. A non-transitory computer readable storage medium for displaying information to a user, the medium comprising an executable program which when executed by a computer processor causes the computer processor to:

make a therapeutic agent delivery component determination based on data received from a therapeutic agent delivery device or component thereof.

129. The non-transitory computer readable storage medium according to Clause 128, wherein the therapeutic agent delivery component determination comprises therapeutic agent delivery component historical information.
130. The non-transitory computer readable storage medium according to Clause 129, wherein the therapeutic agent delivery component determination is configured to be employed in a therapeutic agent delivery device control process.
131. The non-transitory computer readable storage medium according to Clause 130, wherein the therapeutic agent delivery device control process controls actuation of a delivery device based on the therapeutic agent delivery component determination.
132. The non-transitory computer readable storage medium according to any of Clauses 128 to 131, wherein the therapeutic agent delivery component determination comprises usage information.
133. The non-transitory computer readable storage medium according to Clause 132, wherein the therapeutic agent delivery component determination is configured to be employed in an inventory management process.
134. The non-transitory computer readable storage medium according to Clause 132, wherein the therapeutic agent delivery component determination is configured to be employed in a billing process.
135. The non-transitory computer readable storage medium according to Clause 132, wherein the therapeutic agent delivery component determination is configured to be employed in a reimbursement process.
136. The non-transitory computer readable storage medium according to Clause 132, wherein the therapeutic agent delivery component determination is configured to be employed in a patient management process.
137. A kit comprising:

a therapeutic agent delivery component comprising an identifier; and

sterile packaging.

138. The kit according to Clause 137, wherein the kit comprises a plurality of therapeutic agent delivery components.
139. The kit according to any of Clauses 137 to 138, wherein the kit further comprises a therapeutic agent composition.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1-15. (canceled)

16. An active agent delivery device for delivering a therapeutic agent to a target tissue delivery site, the device comprising:
(a) an active agent delivery component comprising an active agent delivery system comprising a needle; and
(b) an actuator component comprising:
(i) a body having a proximal end and a distal end;
(ii) an active agent delivery system actuator configured to actuate the active agent delivery system; and
(iii) a cooling system comprising a cold element configured to maintain a temperature of the target tissue ranging from about +5 to −80° C.;
wherein the active agent delivery component is release-ably engaged to the distal end of the actuator component.

17. The device according to claim 16, wherein the cooling system is configured to mitigate pain at the target tissue delivery site.

18. The device according to claim 16, wherein the cooling system comprises a low-temperature substance.

19. The device according to claim 16, wherein the actuator component comprises a motor.

20. The device according to claim 16, wherein the device is configured to deliver a therapeutic agent.

21. The device according to claim 20, wherein the therapeutic agent is a VEGF antagonist.

22. The device according to claim 20, wherein the therapeutic agent is a complement inhibitor.

23. The device according to claim 20, wherein the therapeutic agent is delivered in a active agent composition.

24. The device according to claim 23, wherein the active agent composition has a viscosity ranging from 1 to 10,000,000 cps.

25. The device according to claim 23, wherein the active agent composition is a semi-solid.

26. The device according to claim 16, wherein the target tissue is ocular tissue.

27. The device according to claim 16, wherein the active agent delivery component comprises an identifier and the actuator component comprises an identifier reader.

28. The device according to claim 16, wherein the actuator component comprises a communications module.

29. A system comprising:
a device according to claim 16; and
a docking station comprising a cooling system.

30. The system according to claim 29, wherein the cooling system is selected from a group consisting of thermoelectric cooling systems, liquid evaporation cooling systems, solid sublimation cooling systems, Joule-Thompson cooling systems, thermodynamic cycle cooling systems, endothermic reaction cooling systems and a low-temperature substance cooling system.

31. The system according to claim 29, wherein the docketing station is configured to recharge a power source of the actuator component.

32. A docking station for a device according to claim 16, wherein the docking station comprises a cooling system.

33. A method of delivering an active agent to a target tissue delivery site, the method comprising employing a device according to claim 16, to deliver the active agent to the target tissue delivery site.

34. The method according to claim 33, wherein the target tissue delivery site comprises an ocular tissue delivery site.

35. The method according to claim 34, wherein the method is a method of treating a subject for an ocular disease.
